(19) United States
(12) Patent Application Publication
(10) Pub. No.: US 2025/0195725 A1
(43) Pub. Date: Jun. 19, 2025

(54) DRUG DEPOT-BEARING MEDICAL GRAFT IMPLANTS, AND RELATED METHODS AND COMPONENTS

(71) Applicant: Cook Biotech Incorporated West Lafayette, West Lafayette, IN (US)

(72) Inventors: Bhavin B. Shah, West Lafayette, IN (US); Michael C. Hiles, Lafayette, IN (US)

(73) Assignee: Cook Biotech Incorporated, West Lafayette, IN (US)

(21) Appl. No.: 18/956,313

(22) Filed: Nov. 22, 2024

Related U.S. Application Data
(60) Provisional application No. 63/601,960, filed on Nov. 22, 2023.

(51) Int. Cl. A61L 27/54; A61L 27/36

(52) U.S. Cl. CPC A61L 27/54; A61L 27/3633; A61L 2300/216; A61L 2300/406

(57) ABSTRACT

Described are implantable medical pocket devices that include one or more polymeric drug depot structures, in some forms received in a chamber defined between first and second layer components of a wall structure(s) of the pocket device, or in some forms attaching first and second pocket sidewalls of the pocket devices. Certain pocket devices can be prepared by methods involving selective lamination of the layer components to form chamber regions, insertion of the depot structure(s) into the chamber regions, and securing the depot structure(s). Also described are methods of use of the pocket devices.

### CROSS REFERENCE TO RELATED APPLICATIONS

This application claims the benefit of U.S. Provisional Application No. 63/601,960 filed Nov. 22, 2023 entitled DRUG DEPOT-BEARING MEDICAL GRAFT IMPLANTS, AND RELATED METHODS AND COMPONENTS, which is hereby incorporated by reference in its entirety.

### BACKGROUND

Aspects of the present disclosure relate generally to medical graft implants that carry one or more drug depots, and more specifically to medical graft implants comprising decellularized extracellular matrix tissue and having one or more drug depots associated with the decellularized extracellular matrix tissue.

As further background, a variety of medical graft implants are known that incorporate a decellularized extracellular matrix (d-ECM) tissue material. In some cases, d-ECM tissue materials have been isolated from a suitable tissue source from a warm-blooded vertebrate, e.g., from the submucosal, dermal or other tissue of a mammal. Such isolated d-ECM tissue materials, for example, small intestinal submucosa (SIS), can be processed so as to have remodelable properties and promote cellular invasion and ingrowth. Illustratively, d-ECM tissue materials have been suggested and used to form hernia repair grafts, wound dressings, pocket implant structures configured to receive another medical material or implant device (e.g. an electronic device such as a defibrillator, pacemaker or neurostimulator), and others. At times, it is desirable to associate a drug with the medical graft implant, for example carried by a drug depot.

There remain needs for improved and/or alternative medical graft implants with one or more associated drugs, as well as methods for preparing and utilizing them. Desirably, the implants will function beneficially at their implant sites and effectively release the drug, and/or the methods for preparing them will be relatively efficient and economical in manufacture.

### SUMMARY

In some aspects, the present disclosure relates to methods for making a drug depot-bearing medical implant. The methods include providing a first multilayer structure in which a first decellularized extracellular matrix (d-ECM) tissue layer is interfacially bonded to a second d-ECM tissue layer in selected regions to provide a first sidewall chamber between unbonded regions of the first and second d-ECM tissue layers, wherein the first multilayer structure defines an opening to the first sidewall chamber. The method also includes providing a first polymeric drug depot structure in the first sidewall chamber, for example by inserting a first polymeric drug depot structure is through the opening and into the first sidewall chamber. The methods can also include securing the first polymeric drug depot structure in the first sidewall chamber. The securing can include closing the opening, for example by applying a stitch line across the opening. The stitch line can entirely circumferentially surround the first polymeric drug depot structure. The securing may alternatively or in addition include adhering the first polymeric drug depot structure to the first d-ECM tissue layer and/or the second d-ECM tissue layer. In some aspects, the multilayer structure can be prepared by a method that includes (i) providing a multilayer layup including the first d-ECM tissue layer a wetted state and the second d-ECM tissue layer in a wetted state in an overlapped condition, the layup having a first layup region in which a first surface portion of the first d-ECM tissue layer is separated from the first face portion of the second d-ECM tissue layer by a spacer member in a position therebetween, a second layup region in which a second surface portion of the first d-ECM tissue layer is received against a second surface portion of the second d-ECM tissue layer, and a third layup region in which a third surface portion of the first d-ECM tissue layer is received against a third surface portion of the second d-ECM tissue layer, wherein the first layup region is between the second layup region and the third layup region; (ii) drying the multilayer layup so as to dehydrothermally bond the second surface portions to one another and the third surface portions to one another while leaving the first surface portions unbonded to one another; and (iii) removing the spacer member from the position between the first face portion of the first layer and the first face portion of the second layer. The method can also include forming an implantable medical pocket device including a first pocket sidewall, a second pocket sidewall, a pocket chamber between the first pocket sidewall and the second pocket sidewall, and a pocket opening to the pocket chamber, wherein the first pocket sidewall includes at least a first portion of the multilayer structure that includes the first sidewall chamber and the first polymeric drug depot structure.

In other aspects, the present disclosure relates to preparation medical pocket devices. The devices include a first pocket sidewall, a second pocket sidewall, and a pocket chamber between the first pocket sidewall and the second pocket sidewall. The devices also include a first polymeric drug depot structure. In some forms, the first polymeric drug depot structure is positioned in a first sidewall chamber of the first pocket sidewall between a first decellularized extracellular matrix (d-ECM) tissue layer and second d-ECM tissue layer interfacially bonded to one another in selected regions to provide the first sidewall chamber between unbonded regions of the first and second d-ECM tissue layers. Such devices can include a stitch line closing an opening to the first sidewall chamber to secure the first polymeric drug depot structure in the first sidewall chamber. The stitch line can entirely circumferentially surround the first polymeric drug depot structure. The first polymeric drug depot structure can be adhered to the first d-ECM tissue layer and/or the second d-ECM tissue layer to secure it in the first sidewall chamber, additional to or as an alternative to any securing stitch line(s). The first d-ECM tissue layer can be interfacially bonded directly against and to the second d-ECM tissue layer in the selected regions by dehydrothermal bonding. The second pocket sidewall can also include a multilayer structure in which first and second d-ECM tissue layers are interfacially bonded to one another in selected regions to provide a second sidewall chamber between unbonded regions of the first and second d-ECM tissue layers, and a second polymeric drug depot structure can be positioned in the second sidewall chamber. The pocket devices can include stitch line extending in a stitch path and attaching the first pocket sidewall to the second pocket sidewall to create the pocket chamber. In other forms, the first polymeric drug depot attaches the first pocket sidewall to the second pocket sidewall. The pocket devices can have another medical implant, for example an electronic medical implant such as a cardiac pacemaker, a cardiac defibrillator, or a neurostimulator, received in the pocket chamber.

In further aspects, the present disclosure relates to methods for preparing implant combinations for insertion in a patient. The methods include inserting a medical implant into the pocket chamber of an implantable medical pocket device as described above or elsewhere in the present disclosure. The medical implant can be an electronic medical implant such as a cardiac pacemaker, a cardiac defibrillator, or a neurostimulator.

In further aspects, the present disclosure relates to methods for treating a patient that include inserting into an implant site in the patient an implantable medical pocket device as described above or elsewhere in the present disclosure having a medical implant received in the pocket chamber of the pocket device. The implant site can be a subcutaneous implant site. The medical implant can be an electronic medical implant such as a cardiac pacemaker, a cardiac defibrillator, or a neurostimulator.

Additional aspects of the present disclosure, as well as features and advantages thereof and of the above-discussed aspects, will be apparent to those skilled in the pertinent field from the descriptions herein.

### BRIEF DESCRIPTION OF THE FIGURES

FIG. 1 provides a plan view of layer components and a spacer member combined in preparing a sheet layup in accordance with one embodiment herein.

FIG. 2 provides an assembled view of the sheet layup resultant of components of FIG. 1.

FIG. 3 provides a cross-sectional view taken along line 3-3 of FIG. 2 and viewed in the direction of the arrows.

FIG. 4A provides a left end view of a laminate sheet product prepared from the sheet layup of FIG. 2 and FIG. 3, after removal of the spacer member.

FIG. 4B provides a plan view of the laminate sheet product of FIG. 4A and also polymeric drug depot structures to be combined therewith by insertion into a chamber region (shown in phantom) occurring between layers of the laminate sheet product.

FIG. 5 provides a plan view of the laminate sheet product of FIG. 4 with the polymeric drug depot structures (shown in phantom) inserted into the chamber region.

FIG. 6 provides a plan view of the laminate sheet product of FIG. 5 after the application of stitch lines circumferentially surrounding the inserted polymeric drug depot structures.

FIG. 7 provides a plan view of the laminate sheet product of FIG. 6 after folding the product lengthwise to position the left edge shown in FIG. 6 over the right edge shown in FIG. 6 (see FIG. 6, arrows), thereby overlapping a first portion of the laminate sheet product with a second portion of the laminate sheet product.

FIG. 8 provides a plan view of the laminate sheet product of FIG. 7 after the application of a stitch line attaching the first portion of the laminate sheet product to the second portion of the laminate sheet product along a path to create a pocket chamber between the first and second portions.

FIG. 9 provides a plan view of the laminate sheet product of FIG. 8 after cutting to provide a peripheral shape of the product and to create a plurality of through holes through the first and second portions of the laminate sheet product.

FIG. 10 provides a cross-sectional view taken along line 10-10 of FIG. 9 and viewed in the direction of the arrows.

FIG. 11 provides a plan view of the laminate sheet product of FIG. 9 and FIG. 10 having a medical implant inserted in the pocket chamber.

FIG. 12 provides a plan view of another embodiment of a laminate sheet product that can be prepared similarly to the embodiment of FIGS. 1-11, except having additional polymeric drug depot structures.

FIG. 13 provides a plan view of one embodiment of a polymeric drug depot structure.

FIG. 14 provides a side view of the polymeric drug depot structure of FIG. 13.

FIG. 15 provides a cross-sectional view of the polymeric drug depot structure of FIG. 13 taken along line 15-15 and viewed in the direction of the arrows, with a portion thereof also shown in an enlarged view.

FIG. 16 provides an assembled view of an alternative sheet layup for preparing a laminate sheet product in the form of a pocket device.

FIG. 17 provides a plan view of one embodiment of a laminate sheet product in the form of a pocket device preparable from the sheet layup of FIG. 16.

FIG. 18 provides a plan view of another embodiment of a laminate sheet product in the form of a pocket device preparable from the sheet layup of FIG. 16.

FIG. 19 provides an assembled view of another alternative sheet layup for preparing a laminate sheet product in the form of a pocket device.

FIG. 20 provides a plan view of one embodiment of a laminate sheet product in the form of a pocket device preparable from the sheet layup of FIG. 19.

FIG. 21 provides a plan view of another embodiment of a laminate sheet product in the form of a pocket device preparable from the sheet layup of FIG. 19.

FIG. 22 provides a plan view of a laminate sheet product that can be folded in preparing a pocket device and including an applied adhesive material for forming a polymeric drug depot structure and adhering walls of the pocket device along a pocket chamber-forming path.

FIG. 23 provides a plan view of one embodiment of a pocket device preparable from the laminate sheet product of FIG. 22.

FIG. 24 provides a cross-sectional view taken along line 24-24 of FIG. 23 and viewed in the direction of the arrows.

### DETAILED DESCRIPTION

Reference will now be made to embodiments, some of which are illustrated in the drawings, and specific language will be used to describe the same. It will nevertheless be understood that no limitation of the scope of the disclosure is thereby intended. Any alterations and further modifications in the described embodiments and any further applications of the principles as described herein are contemplated as would normally occur to one skilled in the art to which this disclosure relates.

As discussed above, embodiments of this disclosure relate to implantable medical pocket devices, and methods and components for their preparation and use. The medical pocket devices can generally include first and second pocket sidewalls that are connected to one another to form a pocket chamber therebetween. In some forms, the first and/or the second pocket sidewall can include a multilayer structure having first and second decellularized extracellular matrix (d-ECM) tissue layers interfacially bonded to one another in selected regions so as to leave a sidewall chamber between unbonded regions, and wherein a polymeric drug depot structure is received and secured in the sidewall chamber. In other forms, a polymeric drug depot structure attaches the first pocket sidewall to the second pocket sidewall so as to form a pocket chamber between the sidewalls.

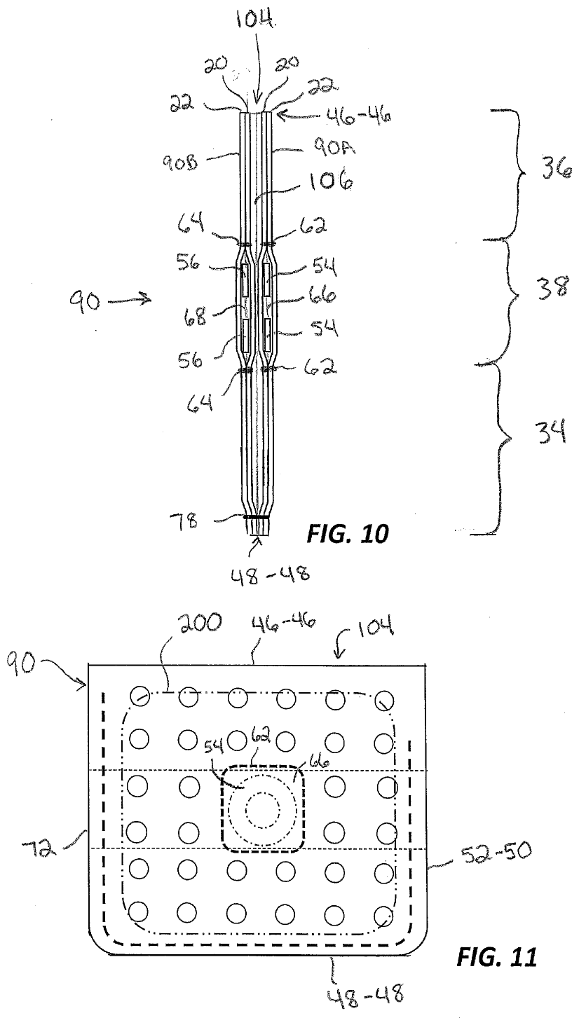

With reference first to FIGS. 9-10, provided are plan and cross-sectional views, respectively, of one illustrative embodiment of an implantable medical pocket device 90 of the present disclosure. Pocket device 90 includes a first pocket sidewall 90A and a second pocket sidewall 90B and a pocket chamber 106 between the first and second pocket sidewalls 90A and 90B. Pocket sidewalls 90A and 90B can exhibit flexibility, for example enabling their conformance to patient tissues when pressed thereagainst. Pocket device 90 also has an opening 104 to the pocket chamber 106, through which a medical implant can be inserted into pocket chamber 106, as discussed further below.

Pocket device 90 has an upper edge 46-46 and a lower edge 48-48, and side edges 52-50 and 72 extending therebetween. As shown, upper and lower edges 46-46 and 48-48 can extend generally parallel to one another, and side edges 52-50 and 72 can extend generally parallel to one another. This arrangement can be incorporated in a generally rectangular peripheral shape of the pocket device 90. The rectangular peripheral shape may for example be a square shape or another rectangular shape, e.g. a rectangular shape in which the side edges 52-50 and 72 are longer than the upper and lower edges 46-46 and 48-48 and the pocket chamber 106 is deeper (considered in the direction from the upper edge 46-46 to the lower edge 48-48) than it is wide (considered in the direction from the side edge 52 to the side edge 72). It will be understood that medical implant pocket devices herein may have other peripheral shapes, including for another polygonal shape or a generally circular or ovate shape, and may have straight peripheral edges, curved peripheral edges, or combinations thereof.

Sidewall 90A is connected to sidewall 90B along a path to define boundaries of the pocket chamber 106 therebetween. In the illustrated embodiment, a stitch line 76, formed with an elongate stitching material such as a suture material, connects sidewall 90A to sidewall 90B along a stitch path to define boundaries of the pocket chamber 106. The stitch line 76 may as examples be a continuous or interrupted stitch line. The stitching material may be a monofilament or multifilament stitching material. The stitching material in particularly advantageous embodiments is a bioabsorbable stitching material, such as a bioabsorbable suture material. For these purposes the suture or other stitching material may be comprised of or formed from a bioabsorbable synthetic polymer, for example a polycaprolactone polymer, a polygalactin polymer, a polyglycolic acid polymer (including copolymers), a polydioxanone polymer, a polylactic acid polymer, a polylactide polymer, a polylactic acid/glycolic acid copolymer, or a combination thereof; or a naturally-occurring material such as a naturally-occurring collagenous material, including as examples d-ECM tissue materials as described herein; or combinations thereof. In other embodiments, the suture or other stitching material can be comprised of or formed from a non-bioabsorbable (i.e. “persistent”) synthetic polymer, for example a polypropylene polymer, a polyester polymer, a polyethylene polymer, an acrylic polymer, a polyamide polymer, an aramid polymer, a fluoropolymer, a fluorocarbon polymer, or a combination thereof. These and other bioabsorbable or non-bioabsorbable materials for use in or for the suture or other stitching material will be apparent to those skilled in the field.

In the illustrated embodiment, the stitch line 76 includes a lower stitch line portion 78 that extends between a first side stitch line portion 80 and a second side stitch line portion 82. Stitch line portion 78 can extend in a direction that is transverse to (e.g. generally perpendicular to) the direction along which the first side stitch line portion 80 extends and/or the direction along which the second side stitch line portion 82 extends. First side stitch line portion 80 can have a first upper terminus 80A spaced a first distance from upper edge 46-46 and second side stitch line 82 can have a second upper terminus 82A spaced a second distance from upper edge 46-46, where the second distance can be greater than the first distance. This latter arrangement can provide an opening flap region (top right, FIG. 9) incorporating lengths of the side edge 52-50 and upper edge 46-46 along which pocket sidewalls 90A and 90B are not connected to one another, which can be ergonomically beneficial to a user. As shown, the first side stitch line portion 80 can extend inward of and generally parallel to the side edge 72, the second side stitch line portion 82 can extend inward of and generally parallel to the side edge 52-50, and the lower stitch line portion 78 can extend inward of and generally parallel to the lower edge 48-48. As well, the side edge 72 can adjoin the lower edge 48-48 through a first radiused corner edge 94, and the side edge 52-50 can adjoin the lower edge 48-48 through a second radiused corner edge 98. In some forms, the first side stitch line portion 80 can adjoin the lower stitch line portion 78 through a first radiused stitch line corner 96 that is inward of and extends generally parallel to the first radiused corner edge 94, and/or the second side stitch line portion 82 can adjoin the lower stitch line portion 78 through a second radiused stitch line corner 100 that is inward of and extends generally parallel to the second radiused corner edge 98.

While in the illustrated embodiment a stitch line 76 along a path is used to connect pocket sidewall 90A to pocket sidewall 90B to form the pocket chamber 106, in other embodiments another type of connection may be used for these purposes, in addition to or as an alternative to a stitch line. As examples, the connection along the path may be completely or partially provided by adhering pocket sidewall 90A to pocket sidewall 90B, including for example by a biocompatible adhesive that interfacially bonds sidewall 90A to sidewall 90B and/or by dehydrothermally bonding sidewall 90A to sidewall 90B (for instance using lyophilization or vacuum-pressing conditions as discussed herein). These and/or other connections between sidewall 90A and sidewall 90B can be suitably incorporated in the pocket device 90.

In the illustrated embodiment, pocket sidewall 90A and pocket sidewall 90B both include a multilayer structure having a first d-ECM tissue layer interfacially bonded to a second d-ECM tissue layer in selected regions so as to provide a sidewall chamber (sidewall 90A-66; sidewall 90B-68) between unbonded regions of the first and second d-ECM tissue layers. As shown, the multilayer structure can include first layer component 20 in an overlapped relationship with a second layer component 22, wherein the first d-ECM tissue layer can constitute the first layer component 20 or can be one of multiple (e.g. 2 to 4) overlapped and preferably interfacially bonded layers thereof, and the second d-ECM tissue layer can constitute the second layer component 22 or can be one of multiple (e.g. 2 to 4) overlapped and preferably interfacially bonded layers thereof. In some forms, the layer component 20 will include or be constituted of two to four d-ECM layers, including the first d-ECM layer, and/or the layer component 22 will include or be constituted of two to four d-ECM layers, including the second d-ECM layer. Thus, the multilayer structure of pocket sidewall 90A and the multilayer structure of pocket sidewall 90B may have and additional d-ECM layer(s) interfacially bonded to either or each of the first d-ECM tissue layer and the second d-ECM tissue layer. The layer component 20 will have the same number of d-ECM tissue layers as the layer component 22 in beneficial embodiments. As will be discussed further below, layer component 20 of sidewall 90A and layer component 20 of sidewall 90B can originate from and/or be connected as a single continuous layer component piece, and, similarly, layer component 22 of sidewall 90A and layer component 22 of sidewall 90B can originate from and/or be connected as a single continuous layer component piece. However, it will be understood that this is not necessary to the broader aspects of the present disclosure, and the respective layer components 20 of the sidewalls 90A and 90B may not originate from or be connected as a single continuous layer component piece and/or the layer components 22 of sidewalls 90A and 90B may not originate from or be connected as a single continuous layer component piece.

With continuing reference to FIGS. 9-10, the multilayer structures of pocket sidewall 90A and pocket sidewall 90B will now be further discussed. As disclosed above, the multilayer structures include interfacial bonding between the first and second d-ECM tissue layers in selected regions, while other regions are unbonded to provide sidewall chambers for receiving polymeric depot structures. The illustrated embodiment 90 includes a region 38 in which the first and second d-ECM layers are unbonded to one another, positioned between regions 34 and 36 in which the first and second d-ECM layers are interfacially bonded to one another. In the illustrated embodiment the unbonded region 38 extends to the side edge 52, and side edge 52 provides an opening to the defined sidewall chamber region at the side edge 52. Preferred arrangements include sidewall chamber regions, an opening to which is presented at one or more peripheral edges of the pocket device 90. This provides a convenient location for the insertion of polymeric drug depot structure(s) as discussed herein prior to the application of any stitch line(s) or other connections that close the opening, e.g. stitch line 76, stitch line 62, or stitch line 64. As well, in some forms, the unbonded region 38 extends to the side edge 72 and side edge 72 can provide an opening to the defined sidewall chamber region at the side edge 72. In other forms, side edge 72 is formed at a fold of an overall multilayer structure that provides the multilayer structures of both pocket sidewall 90A and pocket sidewall 90B, and side edge 72 does not present an opening to the defined chamber region. The illustrated unbonded region 38 is elongate in a direction from the side edge 72 to the side edge 52-50 of the pocket device 90.

In the illustrated form, the interfacially bonded regions 34 and 36 flank the unbonded region 38 on either side thereof and provide outer boundaries of the sidewall chambers 66 and 68. The interfacially bonded region 36 extends to the upper edge 46-46 of the pocket device 90 in the multilayer structures of both pocket sidewalls 90A and 90B. This can provide a bonded laminate upper edge to each of sidewalls 90A and 90B, with opening 104 occurring between these two bonded laminate upper edges. Opening 104 can thus be the only inter-layer opening presented at the upper edge 46-46 of the pocket device 90 (e.g. as opposed to an arrangement in which the first and second d-ECM tissue layers, and when included any other layer(s) of the layer components 20 and 22, are unbonded at the upper edge 46-46). This can provide beneficial use properties to the pocket device 90, where there is less risk that a user will insert or attempt to insert a medical implant into an opening other than that intended. The interfacially bonded regions 34 and 36 extend from side edge 72 to side edge 52-50 and are elongate in a direction from side edge 72 to side edge 52-50.

Pocket sidewall 90A includes sidewall chamber 66 having first polymeric drug depot structure 54 therein, and pocket sidewall 90B includes sidewall chamber 68 having second polymeric drug depot structure 56 therein. As illustrated, a stitch line 62 applied through the multilayer structure of pocket sidewall 90A, but not through the multilayer structure of pocket sidewall 90B, circumferentially surrounds the first polymeric depot structure 54; and, a stitch line 64 applied through the multilayer structure of pocket sidewall 90B, but not through the multilayer structure of pocket sidewall 90A, circumferentially surrounds the second polymeric depot structure 56. Stitch lines 62 and 64 secure the first and second depot structures 54 and 56 in their respective chambers 66 and 68, and preferably also closely circumscribe the outer peripheries of depot structures 54 and 56 so as to secure the position thereof in pocket sidewalls 90A and 90B, respectively. For example, stitch lines 62 and 64 can be applied circumferentially around the respective peripheries of depot structures 54 and 56 so as to have a maximum spacing therefrom of about 10 mm or less, or about 5 mm or less, and in some forms in the range of about 2 mm to about 10 mm or about 2 mm to about 5 mm. Stitch line 62 and/or 64 or other stitch line(s) incorporated to secure depot structure 54 and/or 56 may not penetrate the depot structure 54 and/or 56, which is considered beneficial, although in other variants such securing stitch line(s) may penetrate the depot structure 54 and/or 56. The stitching material for stitch lines 62 and 64 may for example be any of those identified herein for stitch line 76, or any other suitable material. Further, additional or alternative to the use of a stitching material, another material can be used (e.g. in the same locations/configurations as the stitch lines) to close the chambers 66 and 68 to prevent escape of the depot structures 54 and/or 56. For example, in some forms, a biocompatible adhesive material can be used to bond the first d-ECM tissue layer to the second d-ECM tissue layer to close the chamber 66 and/or 68. The biocompatible adhesive material is desirably an absorbable adhesive material, and may for example be comprised of polyvinyl pyrrolidone polymer, polyvinyl alcohol polymer, polyvinyl acetate polymer, vinyl acetate ester polymer, a polylactic acid polymer, a polylactide polymer, a polylactic acid/glycolic acid copolymer, starch, dextrin, collagen, gelatin, sugar, sugar alcohol, or another material, or combinations thereof. In addition to, or as an alternative to, securing the depot structures 54 and/or 56 within their respective chambers 66 and 68 by stitching closed or otherwise closing the chamber 66 and/or 68, they may be secured therein by any other suitable means. In some embodiments herein, the depot structure 54 and/or 56 can be adhered to a wall surface or surfaces bounding the chamber 66 and/or 68, for example a surface of the first d-ECM tissue layer and/or a surface of the second d-ECM layer bounding the chamber 66 and/or the chamber 68. Adherence of the depot(s) 54,56 to the wall surface(s) can be provided by a biocompatible adhesive material (for example any of those listed hereinabove), by melt bonding wherein a surface portion of the polymeric depot(s) 54,56 is heated to melt or soften a polymeric material of the depot(s) 54,56 and then cooled in contact with the wall surface(s) to solidify and bond the depot(s) 54,56 to the surface(s), by solvent bonding wherein a surface portion of a polymeric material of the polymeric depot(s) 54, 56 is solubilized by contact with a solvent and then the solvent is removed (e.g. evaporated) to solidify the polymeric material in contact with the wall surface(s) to bond the depot(s) 54,56 to the wall surface(s), or another suitable technique. In some embodiments herein, the depot structure 54 and/or 56 or other depot structure(s) herein, as secured in place in a chamber, will have an outer periphery that is at least partially surrounded by, and in some forms completely surrounded by, unbonded regions of the first and second d-ECM tissue layers. As will be appreciated, this can result from positioning the polymeric depot(s) 54,56 in a chamber dimensioned larger than the depot(s) 54,56 and securing the depot(s) 54,56 in position, leaving a portion or all of the outer periphery of the depot(s) 54,56 surrounded by unbonded regions of the first and second d-ECM tissue layers of the larger-dimensioned chamber. It will be understood that such relationships can also apply to other depot(s)/chamber(s) of implantable medical devices herein.

Referring particularly to FIGS. 9 and 10, the implantable medical pocket device 90 also includes a plurality of thru-holes 102A extending completely through pocket sidewall 90A and a plurality of thru-holes 102B (not shown in FIG. 9, shown in phantom in FIG. 10) extending completely through pocket sidewall 90B. Thru-holes 102A and thru-holes 102B can facilitate the passage of bodily fluids of a patient through pocket sidewall 90A and pocket sidewall 90B, respectively, when the pocket device 90 is implanted in the patient. This can in turn facilitate the passage of the bodily fluids into and out of the pocket chamber 106 and through the pocket device 90 as a whole. Thru-holes 102A and thru-holes 102B are beneficially circular in shape, as shown, but other shapes may also be used. Thru-holes 102A and through holes 102B can in some forms be corresponding in number and/or corresponding in location so as provide straight passageways extending entirely through pocket device 90 (when there is nothing received within the pocket chamber 106). Thru-holes 102A and 102B can be die-cut together to achieve such an arrangement, as an example. In preferred embodiments, the thru-holes 102A are positioned such that none of them overlaps the depot structure 54, which is covered on its inwardly-facing side by a continuous intact surface of the first d-ECM layer of the first layer component 20 of pocket sidewall 90A and on its outwardly-facing side by a continuous intact surface of the second d-ECM layer of the second layer component 22 of pocket sidewall 90B; and, the thru-holes 102B are positioned such that none of them overlaps the depot structure 56, which is covered on its inwardly-facing side by a continuous intact surface of the first d-ECM layer of the first layer component 20 of the pocket sidewall 90B and on its outwardly-facing side by a continuous intact surface of the second d-ECM layer of the second layer component 22 of the pocket sidewall 90B. Further, as shown, in certain embodiments none of the thru-holes 102A is positioned within or overlapping with the circumferential stitch line 62 and none of the thru-holes 102B is positioned within or overlapping with the circumferential stitch line 64. While in the illustrated embodiments the thru-holes 102A correspond in location with the thru-holes 102B, it will be understood that in other arrangements some or all of the thru-holes 102A may not correspond in location with the thru-holes 102B, and there may be differing numbers of, shapes of (e.g. ovoid shaped, polygonal shaped or slit-shaped), or sizes of, thru-holes 102A and/or thru-holes 102B.

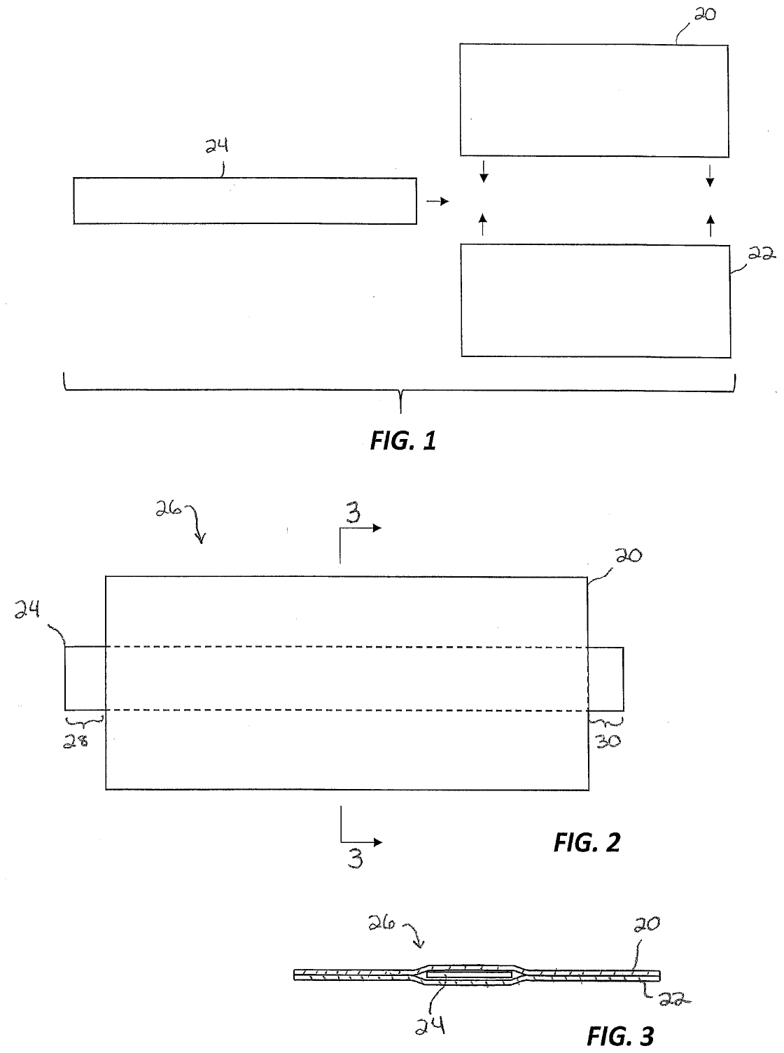

With reference now to FIGS. 1-10 together with FIG. 11, shown in FIG. 11 is the medical implant pocket device 90 having a medical implant 200 received in the pocket chamber 106 thereof, illustrating certain uses of pocket device 90 and combination products that include it. Medical implant may for example be an electronic medical device (e.g. having attached electrical leads that will extend out of pocket device 90) such as a pulse generator of a cardiac pacemaker, a cardiac defibrillator (e.g. an implantable cardioverter-defibrillator, or ICD), or neurostimulator (e.g. for activating or inhibiting the nervous system to reduce pain or to augment, improve or replace function lost to a neurological disease or disorder), although other implants may also be used in conjunction with pocket device 90. Medical implant 200 as received in chamber 106 is surrounded by pocket sidewalls 90A and 90B, which are positioned between outer surfaces of implant 200 and patient tissue at the implant site. Medical implant 200 can be surrounded on all sides by material of the pocket device 90 at the implant site. In some forms, the implant site is a subcutaneous implant site, for example in the chest area of the patient (e.g. for a cardiac electronic medical device), in the abdomen, flank, or buttocks area of the patient (e.g. for a neurostimulator), or any other suitable area. Thus, in some forms, a pocket sidewall (e.g. sidewall 90A or 90B) as implanted in a patient may be received under and against the subcutaneous (deepest) layer of the skin.

Preferred forms of pocket device 90 are provided where the first d-ECM tissue layers of the first and second pocket sidewalls 90A and 90B, and the second d-ECM layers of the first and second pocket sidewalls 90A and 90B, are remodelable d-ECM tissue layers. Such remodelable d-ECM layers can have a porous, collagenous matrix structure that when implanted in a patient leads to the generation of new patient tissue that replaces the collagenous matrix structure as the collagenous matrix structure is absorbed, for example where the collagenous matrix structure is configured to be completely absorbed within six months after implant of the pocket device 90, or within three months after implant of the pocket device 90, for example within a range of about two weeks to about three months after implant of the pocket device 90. In some embodiments herein, the first layer component 20 and the second layer component 22 will be formed entirely of material that is absorbable by the patient, for example where the first layer component 20 is constituted of the first d-ECM tissue layer, as a remodelable d-ECM tissue layer, or is constituted of a laminate (e.g. a dehydrothermally bonded laminate) of the first d-ECM tissue layer, as a remodelable d-ECM tissue layer, and one or more additional remodelable d-ECM layers, for example as described elsewhere herein; and/or the second layer component 22 is constituted of the second d-ECM tissue layer, as a remodelable d-ECM tissue layer, or is constituted of a laminate (e.g. a dehydrothermally bonded laminate) of the first d-ECM tissue layer, as a remodelable d-ECM tissue layer, and one or more additional remodelable d-ECM tissue layers, for example as described elsewhere herein. In these or other forms, the first and second pocket sidewalls 90A and 90B can be entirely replaced by remodeled patient tissue and form a pocket structure of remodeled patient tissue around the implant device 200 or other material received in the pocket chamber 106. In addition, in some forms, all materials of the pocket device 90 (including any stitching material(s) present and any polymeric depot structure(s) present) can be absorbable by the patient, for example within about six months after implant of the pocket device 90, so that the formed pocket structure of remodeled patient tissue is free from any material of the pocket device 90.

In embodiments herein in which the pocket sidewalls 90A and 90B will be replaced by remodeled patient tissue, it is considered advantageous that the pocket sidewalls 90A and 90B have at least portions thereof in which layer component 20 is interfacially bonded to layer component 22. This interfacial bonding can provide a more unitary matrix for cellular invasion and migration through and along the thickness of the pocket sidewalls 90A and 90B during the remodeling process. This can contribute to the development of beneficially formed pocket structures of remodeled patient tissue. In preferred forms, in each of pocket sidewall 90A and pocket sidewall 90B, at least about 30% of the overlapped surface area of layer component 20 and layer component 22 is interfacially bonded, more preferably at least about 50%, and in typical embodiments in the range of about 30% to about 90%, in each case where the remainder of the overlapped surface area is not interfacially bonded (e.g. where the defined chamber(s) as described herein can constitute all or part of such remainder). It is also considered that such overlapped surface area(s) that are not interfacially bonded can provide fluid passageways between layer component 20 and layer component 22 that facilitate the distribution of patient bodily fluid containing dissolved amounts of the drug(s) from the polymeric drug depot(s) present through the pocket sidewalls 90A and 90B. As well, such overlapped surface area(s) that are unbonded can allow for relative movement of the unbonded areas of layer component 20 and layer component 22, e.g. during patient movement, that can further modulate the distribution of patient bodily fluid containing dissolved amounts of the drug(s) from the polymeric drug depot(s) present through the pocket sidewalls 90A and 90B. Thus, the travel of drug-containing patient bodily fluid first through the unbonded passageway areas and then through a porous matrix structure of interfacially bonded areas of layer components 20 and 22, or vice versa, including repeated such occurrences, can contribute to a beneficial distribution of the drug(s) from the polymeric drug depot structure(s) to impregnate the pocket sidewalls 90A and 90B with the drug(s).

In this regard, in advantageous forms, the drug depot(s) of the pocket device 90 will be configured so as to distribute the drug(s) of the depot structure(s) to impregnate all or substantially all (i.e. at least 80%) of the area of pocket sidewalls 90A and 90B. In some forms, the depot structure(s) include at least one antibiotic agent, and such distribution will result in the presence of a minimum inhibitory concentration (MIC) of the antibiotic agent throughout the wall thickness in all or substantially all of the area of pocket sidewalls 90A and 90B within about one day after implantation of the pocket device 90. In certain variants, such MIC preferably can be thereafter sustained at least until the end of three days after implantation, or at least until the end of seven days after implantation, and typically in the range of until the end of three days after implantation to until the end of twenty days after implantation. As well, in some forms, the pocket device 90 will be configured to distribute the drug(s) of the depot(s) to patient tissue adjacent to the implanted pocket device 90, in addition to or as an alternative to distributing the drug to materials of the pocket sidewalls 90A and 90B.

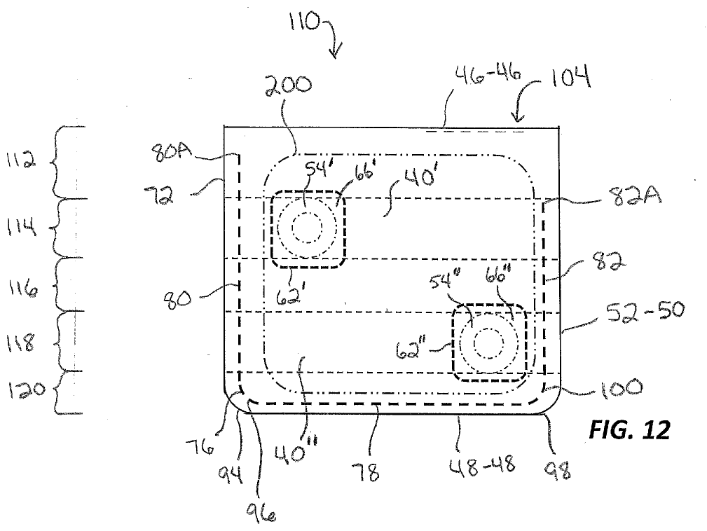

The composition and the number and position of the polymeric drug depot structure(s) can be selected to provide the desired drug distribution properties, for example as discussed above. In this regard, while the pocket device 90 of FIGS. 1-11 includes two polymeric depot structures 54 and 56, a single polymeric drug depot structure or more than two polymeric drug depot structures can be included in other embodiments. Illustratively, FIG. 12 shows a pocket device 110 which is similar in many respects to pocket device 90 discussed above, where like parts are given like numbers to those in FIGS. 1-11. Pocket device 110, however, includes a total of four polymeric drug depot structures, including two polymeric drug depot structures 54′ and 54″ in pocket sidewall 90A, and two corresponding polymeric drug depot structures in pocket sidewall 90B (not illustrated). As examples, the polymeric depot structures 54′ and 54″ can correspond in location to respective ones of the polymeric depot structures in pocket sidewall 90B, or may be positioned in non-corresponding locations. The polymeric drug depot structures 54′ and 54″ are circumferentially entirely surrounded by respective stitch lines 62′ and 62″, and are located in respective chambers 66′ and 66″. The pocket sidewalls 90A and 90B of pocket device 110 include a first interfacially bonded region 112 of layer components 20 and 22, a first non-bonded region 114 of layer components 20 and 222 adjacent to region 112 (providing first chamber region 40′ for receiving polymeric drug depot structure(s)), a second interfacially bonded region of layer components 20 and 22 adjacent to region 114 and opposite to region 112, a second non-bonded region 118 of layer components 20 and 22 adjacent to region 116 (providing second chamber region 40″ for receiving polymeric drug depot structure(s)), and a third interfacially bonded region of layer components 20 and 22 adjacent to region 118 and opposite to region 116. These and other arrangements that accommodate a plurality of polymeric drug depot structures of the pocket devices herein, for example between 2 and 30 polymeric drug depot structures in some forms, will be capable of practice by persons of skill in the field from the descriptions herein.

Any of a wide variety of drugs, or combinations of drugs, may be included in the polymeric drug depot structure(s). The drug(s) can be an antimicrobial agent or a combination to two or more antimicrobial agents. Illustrative antimicrobial agents include, for example, antibiotics such as penicillin, tetracycline, chloramphenicol, minocycline, rifampin, doxycycline, vancomycin, bacitracin, kanamycin, neomycin, gentamycin, erythromycin and cephalosporins. Examples of cephalosporins include cephalothin, cephapirin, cefazolin, cephalexin, cephradine, cefadroxil, cefamandole, cefoxitin, cefaclor, cefuroxime, cefonicid, ceforanide, cefotaxime, moxalactam, ceftizoxime, ceftriaxone, and cefoperazone, and antiseptics (substances that prevent or arrest the growth or action of microorganisms, generally in a nonspecific fashion) such as silver sulfadiazine, chlorhexidine, sodium hypochlorite, phenols, phenolic compounds, iodophor compounds, quaternary ammonium compounds, and chlorine compounds. Still other drugs can be incorporated in the drug depots, alone or in combination with an antimicrobial agent or each other. Such other drugs may include, for example, anti-clotting agents (e.g. heparin), anti-inflammatory agents, anti-proliferative agents (e.g. taxol derivatives such as paclitaxel), inhibitors of tissue adhesions, nonsteroidal anti-inflammatory drugs (NSAIDs), and others. In some preferred forms, the polymeric drug depot structure(s) will include a combination of antibiotic agents including, or consisting of, rifampin and minocycline.

The polymeric material of the polymeric drug depot structures herein can be any suitable polymeric material. As examples, the polymeric material can be any of those discussed herein for the stitch line 76, and absorbable polymeric materials are preferred, with absorbable polylactic acid/glycolic acid copolymers being particularly preferred.

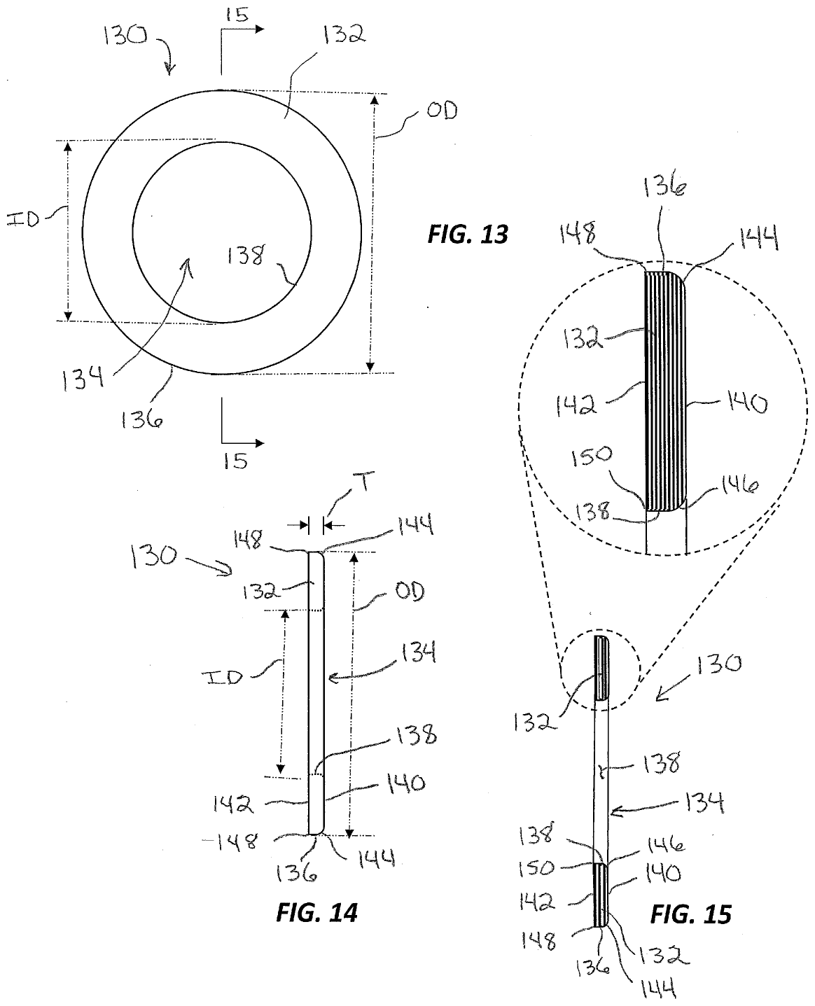

The polymeric drug depot structure(s) can have any suitable shape. As examples, the polymeric drug depot structure(s) can be a material layer having a thickness in the range of about 0.1 mm to about 3 mm, more preferably in the range of about 0.2 mm to about 1 mm, and/or can have a minimum thickness of about 0.1 mm, of about 0.2 mm, or about 0.3 mm. In certain advantageous forms, polymeric drug depot structure(s) can exhibit flexibility, for example enabling their conformance to patient tissues when pressed thereagainst (e.g. along with an associated d-ECM containing layer structure with which it/they are retained). The outer periphery of the material layer of the polymeric drug depot structure(s) can define a circular, ovoid, polygonal, or irregular shape. In some forms, as illustrated in FIGS. 1-11 and discussed further in conjunction with FIGS. 13-15 below, the polymeric drug depot structure(s) will define an inner opening, e.g. providing a ring shape to the depot structure(s). These and other polymeric depot structure(s) as described herein may be incorporated into implantable medical pocket devices as described herein or into other implantable medical devices that incorporate one or more d-ECM layers and/or other bioscaffold material layers or sheets, such implantable medical devices including for instance implantable pockets or pouches, hernia repair or other tissue support devices, reconstructive surgery devices, wound dressing devices, and others.

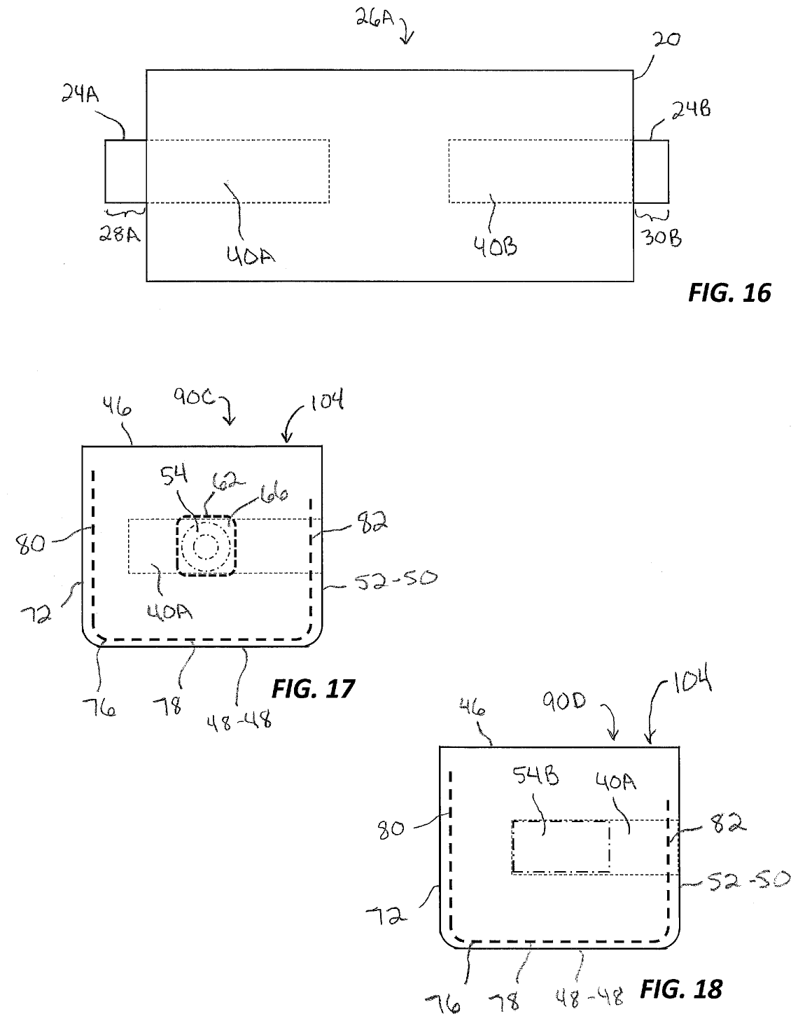

With reference now to FIGS. 1-9, certain embodiments related to methods of preparation, and related structural features, will now be further described. FIG. 1 illustrates components that can be used in the preparation of an implantable medical pocket device, for example pocket device 90, as described herein. These include a first layer component 20, a second layer component 22, and a spacer member 24. The first and second layer components can for example be any of those as described herein. Again, preferred layer components 20 and 22 will each include a d-ECM tissue layer, and in some forms each layer component 20 and 22 will be constituted of or will include a laminate of two or more d-ECM layers interfacially bonded to one another. Layer components 20 and 22 are arranged in an overlapping condition, with the spacer member 24 positioned between them, for instance as in the multilayer product layup 26 illustrated FIGS. 2 and 3. The spacer member 24 can be comprised of any suitable material that prevents contact of the layer component 20 with the layer component 22 in the region in which the spacer member occurs between layer component 20 and layer component 22, and is preferably permeable to water vapor. Spacer member 24 may for example be a layer, such as a film, in some forms having a thickness in the range of about 0.1 mm to about 1 mm, and/or can be made of a water vapor-permeable porous polymeric material such as a porous film of spunbound polyethylene fibers (e.g. a commercially available Tyvek® film product). As shown in FIG. 2, the spacer member 24 can have a length that is greater than that of the layer components 20 and 22 in the region in which spacer member 24 occurs, so that the product layup 26 can include portions 28 and 30 of the spacer member exposed beyond the layer components 20 and 22 on two opposed sides. However, this is not necessary in other embodiments, as the spacer member 24 or multiple spacer members may be positioned so as not to traverse an entire length of the layer components where member 24 occurs, for example with one end of the spacer member(s) positioned at a location between layer components 20 and 24 and another end exposed beyond edges of layer components 20 and 22, and/or with the spacer member(s) entirely located between layer components 20 and 22 (e.g. wherein a layer component 20 and/or 22 can later be cut to create an opening to remove the spacer member(s) from between layer components 20 and 22). Some such alternative embodiments are discussed in conjunction with FIGS. 16-21 below as illustrations. These and other arrangements that will result in unbonded regions between layer components 20 and 22 will be apparent to and workable by those skilled in the field.

It is often beneficial to provide layer components 20 and 22 as in the product layup 26 of FIGS. 2 and 3 in a wetted condition, for example wetted with an aqueous medium which in some forms can be water, for example distilled water or deionized water. This wetting can be carried out before the layer components 20 and 22 are assembled to the configuration shown in FIG. 2, after, or both. In particular embodiments, layer components 20 and 22 are provided in a wetted condition where direct dehydrothermal interfacial bonding of d-ECM tissue layers of layer components 20 and 22 to one another is to be undertaken. In such wetted cases, a step of applying pressure to surfaces of the product layup 26 of FIGS. 2-3 to remove any air bubbles trapped between layer components 20 and 22 can be performed. As one example, pressure can be applied to the surfaces of the product layup 26 to remove any air bubbles by wiping across the surfaces with the edge of an instrument, such as the a blunt edge of a spatula.

The product layup 26, in wetted condition, can be subjected to conditions that dehydrothermally bond a region(s) of a surface (or face) of a d-ECM tissue layer of layer component 20 directly to a region(s) of a surface (or face) of a d-ECM tissue layer of layer component 22. In such dehydrothermal bonding, the surface region(s) of the respective d-ECM layers of layer components 20 and 22 will be in contact with one another while the d-ECM layers, and typically the whole of components 20 and 22, are dried. Suitable methods for dehydrothermal bonding of d-ECM layers to one another are discussed hereinbelow, any of which alone or in combination can be used in this method. The presence of spacer member 24 will prevent regions of the respective d-ECM layers coextensive with the member 24 from dehydrothermally bonding to one another.

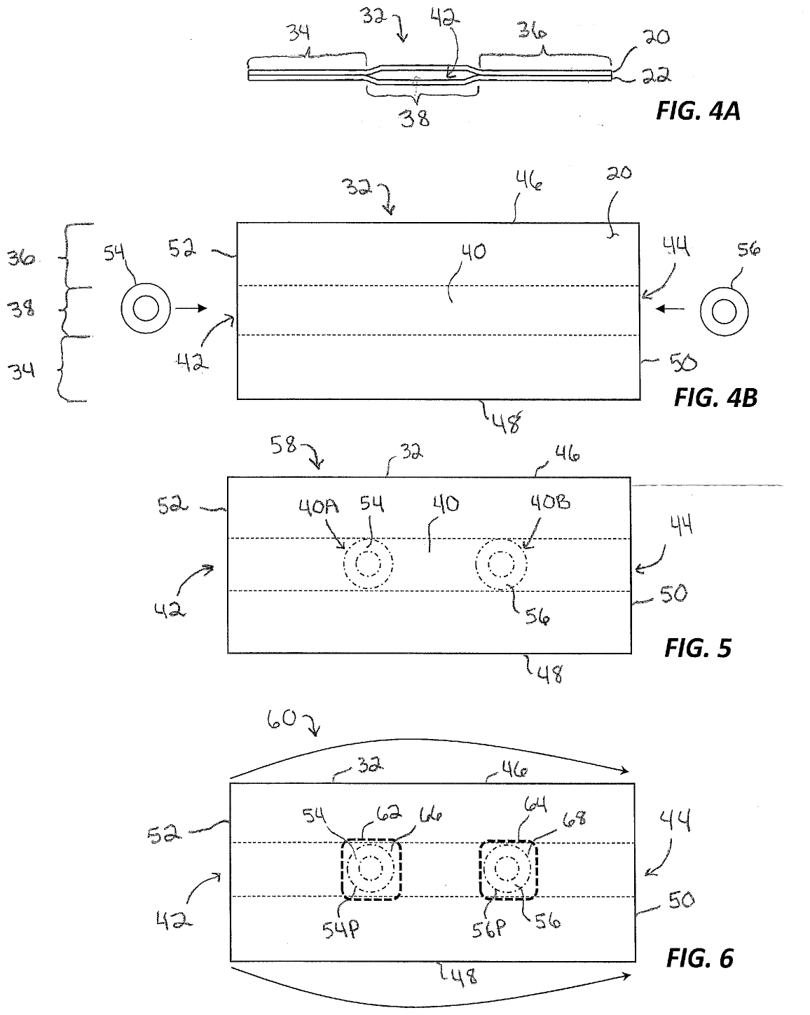

FIG. 4A shows an end view, and FIG. 4B provides a plan view, of a dried multilayer construct 32 after dehydrothermal bonding as discussed above and removal of the spacer member 24. Dried multilayer construct 32 includes a first and second regions 34 and 36 in which respective surfaces of a d-ECM tissue layer of layer component 20 and a d-ECM tissue layer of layer component 22 are dehydrothermally bonded to one another, and a third region 38 in which respective surfaces of the d-ECM tissue layer of layer component 20 and the d-ECM tissue layer of layer component 22 are not bonded to one another to provide a chamber region 40 bounded by unbonded surfaces of the respective d-ECM tissue layers of layer components 20 and 22. The dried multilayer construct has an upper edge 46 opposite of a lower edge 48 and first and second side edges 50 and 52 opposite one another and extending between the upper and lower edges 46 and 48. The third region 38 extends to opposite side edges 50 and 52 of the dried multilayer construct 32, providing openings 42 and 44 for accessing the chamber region 40 at the edges 52 and 50, respectively, of the construct 32. Chamber region 40 in the illustrated embodiment is formed as a channel extending the entire length between edges 52 and 50 of the construct 32. It will be understood that other arrangements providing a chamber or chambers for receiving one, or multiple, polymeric drug depot structure(s), and an opening or openings to access the chamber(s), may also be used in other embodiments herein.

Referring now more specifically to FIG. 4B and FIG. 5 together, also provided are a first polymeric drug depot structure 54 and a second polymeric drug depot structure 56. Drug depot structures 54 and 56 are inserted into the chamber region 40 from opening 42 and/or 44, and moved inwardly from the edge(s) 52 and/or 50 to respective desired positions 40A and 40B within the chamber region 40 of the dried multilayer construct 32. Thus a drug depot-containing dried multilayer construct 58 is formed. Optionally, temporary pins or other temporary securing means can be used to hold depot structures 54 and 56 at their desired positions. Performing this and/or other preparative manipulations of the depot structures 54 and 56 relative to the dried multilayer construct 32, as opposed to having the construct 32 in a condition wetted with a liquid medium (e.g. an aqueous liquid medium), can be particularly beneficial where one or more components of the depot structures 54 and 56, for example one or more drugs contained by the depot structures 54 and 56, is/are soluble in the liquid medium. The dried condition of the construct 32 can prevent mobilization of the drug(s) or other soluble component(s) of the drug depot structures 54 and 56 that would otherwise occur during the preparative manipulation(s). A wide variety of drugs are water soluble, including many antibiotics such as rifampin and/or minocycline, and preparative methods herein can be used with particular advantage in such cases.

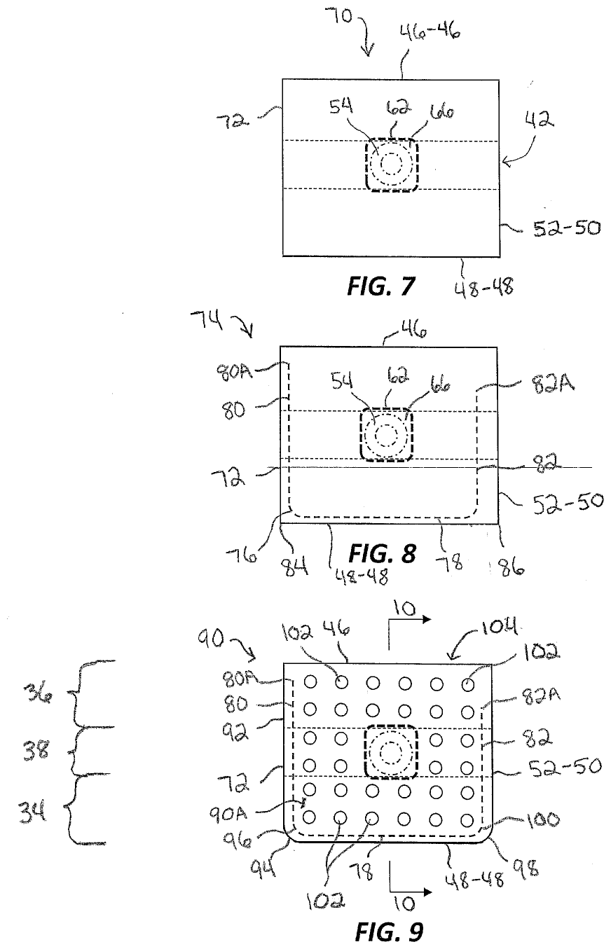

With reference to FIG. 6, in some embodiments, stitch lines 62 and 64 are applied to prevent or limit movement of the drug depot structures 54 and 56 relative to the multilayer construct 32. This provides a stitched, drug depot-containing multilayer construct 60. If used, temporary pins or other temporary means for securing the position of drug depot structures 54 and 56 after the application of stitch lines 62 and 64 or after the application of other materials to prevent or limit movement of depot structures 54 and 56, e.g. with such materials to form parts of the prepared implantable medical pocket device. Stitch lines 62 and 64 can comprise any suitable stitching material, for example any of those described hereinabove, and can be continuous or discontinuous and have any suitable configuration, such as those described hereinabove. The stitching material of stitch lines 62 and 64 preferably extends entirely through the combined thickness of layer component 20 and layer component 22 to connect them together along the stitch lines. Stitch lines 62 and 64 are preferably lock stitch lines. In the illustrated embodiment, stitch lines 62 and 64 completely circumferentially surround drug depot structures 54 and 56, respectively. This can confine the drug depot structure 54 in chamber space 66 and drug depot structure 56 in chamber space 68. With continuing reference to FIG. 6 now also with FIG. 7, in a further and potentially subsequent step, the stitched, drug depot-containing multilayer construct 60 can be folded upon itself to create a sided construct 70 having drug depot structure 54 positioned in a first side and drug depot structure 56 positioned in a second side, for example by positioning side edge 52 overtop and in alignment with side edge 50 (see arrows, FIG. 6). Sided construct 70 will thus have a first side edge 72 (see FIG. 7, left side) formed as a fold of the multilayer construct 32, a second side edge 52-50 opposite thereof formed as an overlap of edges 52 and 50 of the multilayer construct 32, an upper edge 46-46 formed as an overlap of first and second lengths of upper edge 46 of the multilayer construct 32, and a lower edge 48-48 formed as an overlap of first and second lengths of lower edge 48 of the multilayer construct 32.

It will be understood that in other embodiments, folding of the construct 32 to create a sided construct can be performed in other manners that position drug depot structures 54 and 56 in respective sides. For example, such folding operations will not necessarily need to align particular edge portions of the construct with one another as discussed immediately above. This is especially the case where cutting operations (e.g. as discussed below) may be subsequently performed to remove material of the construct 32 to create one or more of the sides of a prepared pocket device structure. In other variations, prior to folding, the drug depot-containing multilayer construct 60 can be cut to provide a desired peripheral shape to a prepared pocket device structure, or to be further cut after folding to create such a desired shape. Still further, a wetted product layup such as product layup 26 (see FIGS. 2 and 3) can be provided in a folded, sided state (optionally with another spacer member positioned between and separating overlapped side surfaces thereof to prevent their bonding to one another), and then dried in the folded, sided state, followed by removal of the spacer member(s) and insertion and positioning of polymeric drug depot structures as discussed in conjunction with FIG. 4B and FIG. 5, and securing the drug depot structures as discussed in conjunction with FIG. 6 or otherwise, to provide a drug depot-containing construct corresponding to that shown in FIG. 7. These and other preparative variations will be apparent to and workable by persons skilled in the field from the descriptions given herein.

Referring now more particularly to FIG. 7 and FIGS. 8-10 together, in a further and potentially next step, a stitch line 76 can be applied to attach the first and second sides of the sided construct 70 to one another and form a pocket chamber 106 between first and second sidewalls 90A and 90B and an opening 104 that provides access to the pocket chamber 106. Stitch line 76 can comprise any suitable stitching material, for example any of those described hereinabove, and can be continuous or discontinuous and have any suitable configuration, such as those described hereinabove. Stitch line 76 is preferably a lock stitch line. The stitching material of stitch line 76 preferably extends through the entire thickness of the first and second sides of the sided construct 70 to connect them together. In the illustrated embodiment, stitch line 76 includes a lower stitch line portion 78 forming a lower boundary of the pocket chamber 106, a first side stitch line portion 80 forming a first side boundary of the pocket chamber 106, and a second side stitch line portion 82 forming a second side boundary of the pocket device opposite the first side boundary. As illustrated, the first side stitch line portion 80 can have an first upper terminus 80A, and the second stitch line portion 82 can have a second upper terminus 82A. In some forms, the first upper terminus 80A can be spaced from the upper edge 46 a first distance, and the second upper terminus 82A can be spaced from the upper edge 46 a second distance that differs from the first distance. The second distance can be greater than the first distance, for example where the second distance is at least about 130% of (i.e. 1.3 times) the first distance, and in some embodiments where the second distance is in the range of about 150% to about 500% of the first distance. Such arrangements can provide a sidedness to the opening 104, where a length of the opening 104 near the upper terminus 82A (top right, FIG. 9) is adjacent an opening flap region incorporating lengths of the side edge 52 and upper edge 46 along which pocket sidewalls 90A and 90B are not connected to one another. In some forms, the first side stitch line portion 80 can adjoin the lower stitch line portion 78 through a first radiused stitch line corner 96 and/or the second side stitch line portion 82 can adjoin the lower stitch line portion 78 through a second radiused stitch line corner 100.

In a further and potentially subsequent, or potentially previous step, a plurality of thru-holes can be provided in pocket sidewall 90A and/or pocket sidewall 90B. The thru-holes can have any suitable size, shape or number, and can be formed by cutting away material of the pocket sidewall 90A and/or pocket sidewall 90B by die cutting or any other suitable blade-based, energy-based (e.g. laser cutting), or other cutting technique. In some preparative embodiments, the thru-holes can be formed prior to forming the folded, multilayer construct 70 of FIG. 7. In other preparative embodiments, the thru-holes can be formed after forming the folded, multilayer construct 70 of FIG. 7. In the illustrated embodiment of pocket device 90, a plurality of circular thru-holes 102 are formed in pocket sidewall 90A and a plurality of circular thru-holes 104 are formed in pocket sidewall 90B in locations corresponding to thru-holes 102. In addition or alternatively, peripheral portions of material can be cut away to form a desired peripheral edge configuration of the pocket device 90. For example, in some forms, excess corner material 84 can be cut away in forming a radiused corner edge 94 adjoining side edge 72 and lower edge 48-48 and excess corner material 86 can be cut away in forming a radiused corner edge 98 adjoining side edge 52-50 and lower edge 48-48. In some forms, this can be done to create a structure wherein first radiused stitch line corner 96 is inward of and extends generally parallel to the first radiused peripheral corner edge 94, and/or the second radiused stitch line corner 100 is inward of and extends generally parallel to the second radiused peripheral corner edge 98. As well, as shown, the first side stitch line portion 80 can be inward of and extend generally parallel to the peripheral side edge 72 and/or the second side stitch line portion 82 can be inward of and extend generally parallel to the peripheral side edge 52-50. It will be understood that in other forms, the peripheral side edge 72 of the prepared pocket device 90 may not be formed as a fold, for example where peripheral material including the previously-present fold is cut away to form a corresponding side edge of the pocket 90, and side edge 52-50 may not be formed of the previously-present side edges 52 and 50 of the formed sided construct (see e.g. FIGS. 7-8), for example where peripheral material including the side edges 52 and 50 is cut away to form a corresponding side edge of the pocket 90.

As discussed above, other arrangements for creating a chamber(s) between layer components 20 and 22, and products obtainable therefrom, are contemplated herein. For example, with reference to FIGS. 16 to 18, shown are an alternative layup of layer components 20 and 22 with spacer members to create chambers, and products producible therefrom. Shown in FIG. 16 is assembled multilayer layup 26A, which can be assembled using steps corresponding to those discussed in conjunction with FIGS. 1 and 2, except using a first spacer member 24A and a second spacer member 24B (instead of single spacer member 24). As shown, spacer members 24A and 24B are positioned so as not to traverse an entire length of the layer components 20 and 22 where members 24A and 24B occur, such that one end of each of the spacer members 24A and 24B is positioned at a location between layer components 20 and 22 and another end exposed beyond edges of layer components 20 and 22. This provides respective exposed portions 28A and 30B of spacer members 24A and 24B, and regions 40A and 40B in which layer components 20 and 22 are separated by the spacer members 24A and 24B, to remain unbonded as corresponding chamber regions in subsequent steps. The multilayer layup 26A can then be used in steps corresponding to those discussed in conjunction with FIGS. 4-10 above to prepare an alternative pocket device 90C as depicted in FIG. 17. Some components of pocket device 90C corresponding to those of pocket device 90 of FIG. 9 are correspondingly numbered in FIG. 17, and it will be understood that other components (numbered or otherwise) of pocket device 90 of FIG. 9 can also be present in pocket device 90C, including for example openings 102 which are not shown in FIG. 17 but can optionally be present.

Referring now to FIG. 18, shown is another embodiment of a pocket device 90D that can be prepared using multilayer product layup 26A of FIG. 16. The preparation of pocket device 90D can proceed the same as device 90C, except instead of inserting depots 54 and applying stitch lines 62 and 64 surrounding depots 54, a first polymeric drug depot structure 54B and a second polymeric drug depot structure 54B are respectively provided in chambers 40A and 40B (depot structure 54B in chamber 40B not illustrated in FIG. 18) in a condition adhered to adjacent surfaces of layer components 20 and 22 providing opposing walls of chambers 40A and 40B. In some forms, prefabricated polymeric drug depot structures 54B can be inserted into chambers 40A and 40B and adhered with a separate biocompatible adhesive to the adjacent surfaces of layer components 20 and 22. In other forms, a flowable, curable depot-forming material containing the polymer(s) and drug(s) to be included in the polymeric drug depot structures 54B can be introduced (e.g. injected through a tubular introducer) into chambers 40A and 40B and then cured to form the polymeric drug depot structures 54B in situ in the chambers 40A and 40B. The flowable, curable depot-forming material can be introduced in a predetermined amount to provide the desired total of the drug(s) to be included in the formed polymeric drug depot structures 54B. The depot-forming material can be curable in any suitable manner, including for example by evaporation of a solvent, by transitioning from a molten state to an unmolten solid state, by crosslinking, or in any other manner described herein for forming a polymeric drug depot structure. The curing of the depot-forming material can cause the formed polymeric drug depot structures 54B to be adhered to the adjacent surfaces of layer components 20 and 22. The in situ formed polymeric drug depot structures 54B can have any suitable shape. In some embodiments, the curable, flowable depot-forming material can be introduced into the chambers 40A and/or 40B so as to extend to all of or a portion of the boundary edge of the chamber defined by laminated regions of the layer components 20 and 22, and thereafter cured, thereby forming polymeric a polymeric drug depot structure 54B having an outer periphery conforming and corresponding in shape to all of or a portion of such boundary edge of the chamber. As shown in FIG. 18, the polymeric drug depot structures of the illustrated embodiment do not occupy the entire chamber 40A (or 40B) between the layer components 20 and 22, and have outer peripheries conforming and corresponding in shape to a portion of the above-described boundary edge of the chamber 40A. While FIG. 18 does not depict a suture line surrounding the polymeric drug depot structure 54B (e.g. corresponding to suture line 62 of FIG. 17), it will be understood that such a suture line could be included if desired.

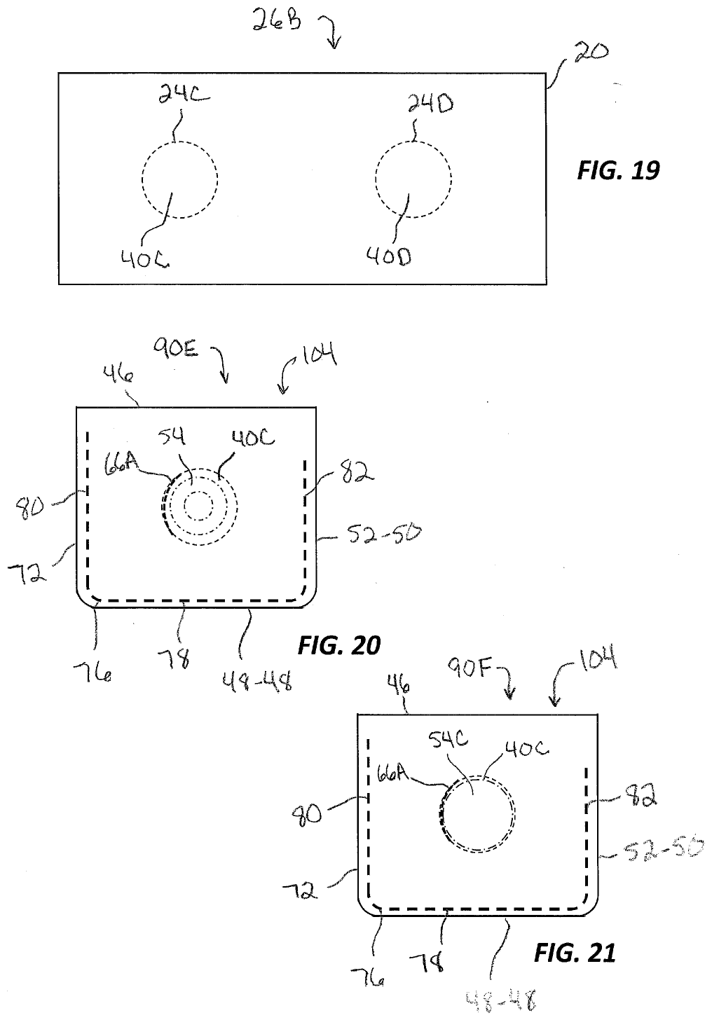

With reference to FIGS. 19 to 21, shown are another alternative layup of layer components 20 and 22 with spacer members to create chambers, and products producible therefrom. Shown in FIG. 19 is assembled multilayer layup 26B, which can be assembled using steps corresponding to those discussed in conjunction with FIGS. 1 and 2, except using a first spacer member 24C and a second spacer member 24D (instead of single spacer member 24). As shown, spacer members 24C and 24D are positioned so as to be entirely between the layer components 20 and 22. This provides regions 40C and 40D in which layer components 20 and 22 are separated by the spacer members 24C and 24D, to remain unbonded as corresponding chamber regions in subsequent steps. The spacer members 24C and 24D as shown are generally circular in shape, although other shapes such as ovoid, polygonal, or irregular shapes, may be used. The multilayer layup 26B can then be used in steps corresponding to those discussed in conjunction with FIGS. 4-10 above, except taking steps to create openings to the formed chambers 40C and 40D and introducing suture lines to close the created openings, to prepare an alternative pocket device 90C as depicted in FIG. 20. In particular, after dehydrothermally interfacially bonding, or otherwise bonding, regions of layer components 20 and 22 of layup 26B not corresponding in location to spacer members 24A and 24B, layer component 20 and/or layer component 22 can be cut in locations corresponding to chambers 40C and 40D to create respective openings to the chambers. The spacer members 24C and 24D can then be removed, followed by insertion of respective polymeric drug depot structures 54 and 56 through the created openings and into chambers 40C and 40D. The created openings can then be closed with suture lines 66A and 66B (the latter not shown in FIG. 20), which can as shown be introduced to close the openings but not entirely circumferentially surround the polymeric drug depot structures 54 and 56 (the latter not shown in FIG. 20). It will be understood that in other forms the suture lines that close the created openings can entirely circumferentially surround the polymeric drug depot structures 54 and 56. Some components of pocket device 90E corresponding to those of pocket device 90 of FIG. 9 are correspondingly numbered in FIG. 20, and it will be understood that other components (numbered or otherwise) of pocket device 90 of FIG. 9 can also be present in pocket device 90E, including for example openings 102 which are not shown in FIG. 20 but can optionally be present.

Referring now to FIG. 21, shown is another embodiment of a pocket device 90F that can be prepared using multilayer product layup 26B of FIG. 19. The preparation of pocket device 90F can proceed the same as device 90E, except instead of inserting depots 54 and 56 through the created openings to chambers 40C and 40D, a flowable, curable depot-forming material containing the polymer(s) and drug(s) to be included in polymeric drug depot structures 54C can be introduced (e.g. injected through a tubular introducer) into chambers 40C and 40D through the created openings and then cured to form the polymeric drug depot structures 54C in situ in the chambers 40C and 40D. The flowable, curable depot-forming material can be introduced in a predetermined amount to provide the desired total of the drug(s) to be included in the formed polymeric drug depot structures 54B. The depot-forming material can be curable in any suitable manner, including for example by evaporation of a solvent, by transitioning from a molten state to an unmolten solid state, by crosslinking, or in any other manner described herein for forming a polymeric drug depot structure. The curing of the depot-forming material can cause the formed polymeric drug depot structures 54C to be adhered to the adjacent surfaces of layer components 20 and 22. The in situ formed polymeric drug depot structures 54C can have any suitable shape. In some embodiments, the curable, flowable depot-forming material can be introduced into the chambers 40C and/or 40D so as to extend to all of or a portion of the boundary edge of the chamber defined by laminated regions of the layer components 20 and 22, and thereafter cured, thereby forming polymeric a polymeric drug depot structure 54C in the subject chamber having an outer periphery conforming and corresponding in shape to all of or a portion of such boundary edge of the chamber. As shown in FIG. 21, the polymeric drug depot structures of the illustrated embodiment occupy essentially the entire chamber 40C (or 40D, not shown in FIG. 21) between the layer components 20 and 22, and have outer peripheries conforming and corresponding in shape to essentially all of the above-described boundary edge of the chamber. While FIG. 21 depicts a suture line 66A closing the created opening to chamber 40C (and a corresponding suture line can be included to close the created opening in chamber 40D, not shown), it will be understood that such a suture line or lines can be omitted in other embodiments.

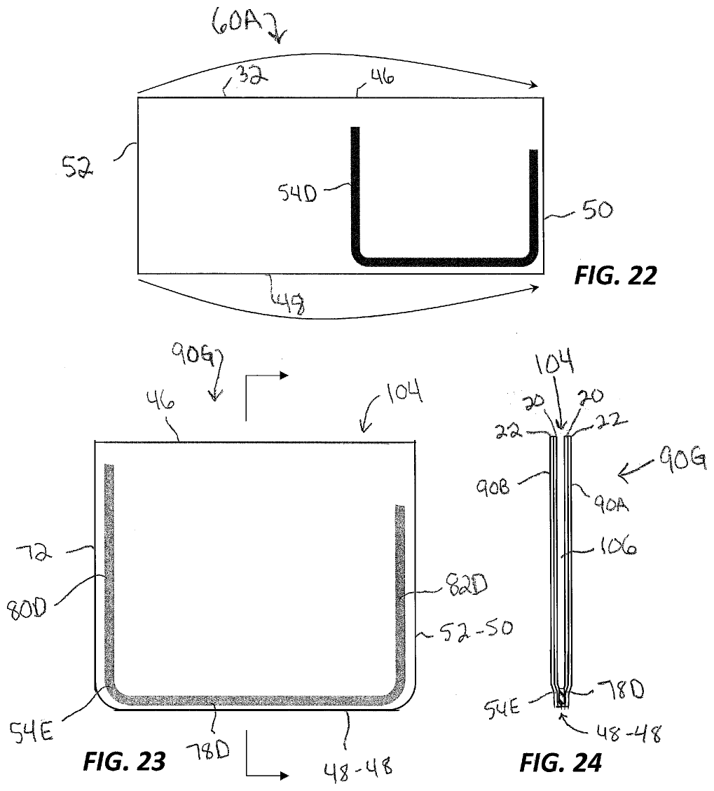

In other embodiments herein, implantable pocket devices will include first and second pocket sidewalls and a pocket chamber between the first and second pocket sidewalls, wherein at least a portion of a path of attachment of the first and second pocket sidewalls to one another to form the pocket chamber is an adhesive attachment provided by one or more polymeric drug depot structures. In some forms, a substantial portion of (i.e. at least 50% of a length of) such path of attachment, or the entirety of such path of attachment, is provided by one or more polymeric drug depot structures, and in some variants by one polymeric drug depot structure. Referring now to FIGS. 22 to 24, in conjunction with FIGS. 1 to 10 and discussions thereof above, certain illustrative embodiments of pocket device products and preparative methods herein will be discussed. First, shown in FIG. 22 is a multilayer construct 60A that can be folded in the preparation of an implantable pocket device. Construct 60A can have layer components 20 and 22 interfacially bonded to one another, and can be prepared in similar fashion to construct 60 of FIG. 6, except that construct 60A need not and in beneficial forms does not have chamber region 40. Layer components 20 and 22 of construct 60A can be interfacially bonded to one another across their entire overlapped surfaces in some embodiments. As well, construct 60A does not include inserted depot structures 54 and 56 or have applied suture lines 62 and 66. Instead, construct 60A has applied a surface thereof selectively along a path configured to create a pocket, a volume of a flowable, curable depot-forming material 54D containing the polymer(s) and drug(s) to be included in a polymeric drug depot structure. For instance, the path can correspond to that described for suture line 76 in other embodiments herein. The construct 60A can be folded upon itself correspondingly to that described for construct 60 of FIG. 6, to form a sided construct having the volume of flowable, curable depot-forming material positioned between and in contact with the sides. This contact can in some modes of operation cause the volume of flowable, curable depot-forming material to flow and spread, for example creating a wider band of the material in the path; however, if present, this contact-based flow and spread will beneficially not widen the band of the material by more than about 100%. The pressure applied during contact, the viscosity of the curable, flowable depot-forming material during contact (e.g. as an uncured or partially cured material upon contact), the volume applied, and/or other factors, can be selected to control the extent of this widening that occurs, if any. The depot-forming material can be caused to cure, in some forms while compressing the sided construct at least along the path in which the depot-forming material occurs, so as to form a polymeric drug depot structure 54E positioned between and adhering the sides of the sided construct to one another. Steps corresponding to those discussed in conjunction with FIGS. 8-10 can then be conducted to form a pocket device 90G (see FIGS. 23-24) in which the polymeric drug depot structure 54E is positioned between and attaches pocket sidewall 90A to pocket sidewall 90B. An inner edge of the polymeric drug depot structure 54E can be directly exposed to and define a peripheral boundary of the pocket chamber 106 (see in particular FIG. 24). It will be understood that similar exposed and boundary-defining inner edge structures can occur in embodiments wherein the polymeric drug depot structure(s) only partially define the attachment path creating the pocket chamber 106 between layer components 20 and 22 and/or wherein multiple polymeric drug depot structures are included in such attachment path. Some components of pocket device 90G corresponding to those of pocket device 90 of FIGS. 9 and 10 are correspondingly numbered in FIGS. 23 and 24, and it will be understood that other components (numbered or otherwise) of pocket device 90 of FIGS. 9 and 10 can also be present in pocket device 90G, including for example openings 102 which are not shown in FIGS. 23 and 24 but can optionally be present.

The preparation of a pocket devices such as pocket devices 90, 90C, 90E or 90F need not utilize a folded construct to provide sidewalls 90A and 90B. For example, sidewalls 90A and 90B can be provided by separate multilayer constructs each including its own layer component 20 and layer component 22 and each forming its own chamber(s) having polymeric drug depot structure(s) correspondingly positioned and secured therein (when present), where in the pocket preparation a stitch line such as stitch line 76 or an adhesive polymeric drug depot structure such as depot structure 540E, or another attachment mechanism, attaches the separate multilayer constructs to one another to form a pocket chamber therebetween such as pocket chamber 106; and wherein other steps taken in the preparation of the prepared pocket device 90 can correspond to those discussed herein. As well, additional structural features that can be formed in preparing and that can be present in the prepared pocket devices are included in above-made discussions of pocket devices, which will be understood without the need to repeat them here. These and other product and preparative variations will be understood given the teachings herein.

As discussed above, the polymeric depot structure(s) included in the pocket devices herein can have any suitable configuration. In preferred forms, the polymeric drug depot structure will comprise a ribbon of polymeric material (e.g. one or a combination of any of those described herein, preferably absorbable polymer(s)) incorporating a drug (e.g. one or a combination of any of those described herein) and extending in a closed path to define an inner opening, with an inner edge of the ribbon bounding the inner opening and an outer edge of the ribbon defining an outer periphery of the drug depot. This can define a generally ring shaped structure. Such polymeric drug depot structures provide further embodiments herein and can for example be used in medical implants as described herein, or otherwise. The shape of such polymeric drug depot structures, including the defined inner opening, is advantageous in that the depot structures can elute drug outwardly from their outer periphery, for example to impregnate implant material there occurring, as well as inward from the inner edge of the ribbon and into their inner opening, for example to impregnate implant material there occurring. The ring or other closed-path ribbon shape thus beneficially distributes the drug on the implantable device for elution. In certain preferred forms, the polymeric material includes or is constituted of a polylactic acid/glycolic acid copolymer, and/or the polymeric material incorporates rifampin, minocycline, or a combination of drugs consisting of or including rifampin and minocycline. The ribbon can have a maximum thickness in the range of about 0.1 mm to about 3 mm, more preferably about 0.2 mm to about 1 mm, and/or a minimum thickness of about 0.1 mm, of about 0.2 mm, or of about 0.3 mm. The thickness of the ribbon can be substantially constant (i.e. within ±10%), or may vary, for example in a range of greater than ±10% but less than ±50%. In addition or alternatively, taking the width of the ribbon as the smallest distance between a point on its inner edge and a point on its outer edge, the ribbon can have a minimum width in the range of about 1 mm to about 15 mm, more preferably about 2 mm to about 10 mm, and in some forms about 3 mm to about 7 mm; and/or a maximum width of about 15 mm or about 10 mm. The width of the ribbon can be substantially constant (i.e. within ±10%), or may vary, for example in a range of greater than ±10% but less than ±50%. The ratio of the maximum width of the ribbon to the maximum thickness of the ribbon can be in the range of about 150:1 to about 5:1, or in the range of about 50:1 to about 20:1. In some forms, the inner edge of the ribbon, around its entire circumference, will extend generally parallel to the outer edge of the ribbon. For example, the inner edge and the outer edge of the ribbon can extend in paths that are generally concentric with one another, for example generally concentric circular paths, generally concentric ovoid paths, or a generally concentric polygonal (e.g. rectangular, such as square) paths. With reference now to FIGS. 13-15, shown is one illustrative embodiment of such a polymeric drug depot structure 130.

Drug depot structure 130 includes a ribbon 132 of polymeric material extending in a generally circular path so as to define an inner opening 134. Drug depot structure 130 has an outer periphery defined by an outer edge 136 of the ribbon 132, and inner opening 134 has an outer periphery defined by an inner edge 138 of ribbon 132. Outer edge 136 extends in a generally circular first path, and inner edge 138 extends in a generally circular second path concentric to the generally circular first path. The ribbon 132 has a substantially constant width. The ribbon can also have a substantially constant thickness over at least a substantial percentage, for example from 80% to 100%, of its width. Radiused corners of ribbon 132, as discussed below, may be present and can result in a varying thickness over a portion or portions of the width of ribbon 132. The outer edge 136 of ribbon 132 defines an outer diameter “OD” of depot structure 130 and the inner edge 138 of ribbon 132 defines an inner diameter “ID” of the depot structure 130. In some forms, the ratio of the OD to the ID of depot structure 130 will be in the range of about 10:1 to about 1.1:1, or in the range of about 7:1 to about 1.1:1, and beneficially in the range of about 2:1 to about 1.25:1 or in the range of about 1.4:1 to about 1.8:1. In addition or alternatively, the OD of the drug depot structure 130 can be in the range of about 5 mm to about 50 mm, or in the range of about 10 mm to about 50 mm, or in the range of about 15 mm to about 35 mm.

Turning now to a discussion of further features of a preferred ribbon 132, which may be incorporated in drug depot structures where the ribbon 132 extends in a generally circular path, or other closed path (e.g. a generally ovoid or polygonal path), ribbon 132 defines a first side surface 140 and an opposite second side surface 142, and a thickness “T” between the first and second side surfaces 140 and 142. Edges 136 and 138 extend between the first and second side surfaces 140 and 142. In some forms, edge 136 can adjoin side surface 140 in a radiused corner 144 and/or edge 138 can adjoin side surface 140 in a radiused corner 146. Edge 136 and/or edge 138 may also adjoin side surface 142 in a radiused corner; however in certain beneficial forms, edge 136 adjoins side surface 142 in a non-radiused corner 148 and/or edge 138 adjoins side surface 142 in a non-radiused corner 150. For example, a non-radiused corner such as corner 148 or corner 150, considered in cross-section as shown in FIG. 15, can occur at the intersection of two straight lines, for instance extending at an angle of about 90 degrees with respect to one another. A radiused ribbon corner (e.g. corners 144 and 146) or a non-radiused ribbon corner (e.g. corners 148 and 150) as described herein can extend around the entire circumference of edge 136 and/or the entire circumference of edge 138.

In particularly beneficial forms, polymeric drug depot structures 132, or other polymeric drug depot structures described herein, can exhibit flexibility, for example enabling their conformance to patient tissues when pressed thereagainst (e.g. along with their associated flexible pocket sidewalls 90A and 90B of pocket devices herein). Additionally, polymeric drug depot structures 132, or other polymeric drug depot structures described herein, can be essentially flat in a relaxed condition, or they can exhibit a non-planar curvature and/or other non-planar features, in a relaxed condition.

Polymeric drug depot structure 132, or other polymeric drug depot structures described herein, can be prepared by any suitable method. For example, they may be prepared by solvent casting or melt casting, and may be cast to the desired configuration or formed (e.g. cut) from larger cast articles (e.g. sheets) to provide the desired configuration for the depot structure. These and other suitable preparative techniques will be recognized and implementable by persons skilled in the art given the teachings herein.

As disclosed above, implantable medical pocket devices herein, and preparative methods herein, involve the incorporation or use of decellularized extracellular matrix (d-ECM) tissue layers. Suitable d-ECM tissue layers herein can be provided by collagenous d-ECM tissue layers. For example, suitable collagenous d-ECM tissue layers include those comprising submucosa, renal capsule membrane, amniotic membrane, dermal collagen (e.g. decellularized dermis such as decellularized porcine or human dermis), dura mater, pericardium, fascia lata, serosa, peritoneum or basement membrane layers, including liver basement membrane layers. Suitable submucosal d-ECM tissue layers for these purposes include, for instance, d-ECM tissue layers that include intestinal submucosa, for example small intestinal submucosa, stomach submucosa (including in some forms forestomach submucosa), urinary bladder submucosa, and uterine submucosa. These or other d-ECM tissue layers can be characterized as membranous tissue layers obtained from a source tissue and decellularized. These d-ECM tissue layers can have a porous matrix comprised of a network of collagen fibers, wherein the network of collagen fibers retains an inherent network structure from the source tissue. In particular aspects, d-ECM tissue layers comprising submucosa (potentially along with other associated tissues) useful in the present invention can be obtained by harvesting such tissue sources and delaminating a submucosa-containing matrix from smooth muscle layers, mucosal layers, and/or other layers occurring in the tissue source, and decellularizing the matrix before or after such delaminating. For additional information as to some of the d-ECM tissue layer materials useful in the present invention, and their isolation and treatment, reference can be made, for example, to U.S. Pat. Nos. 4,902,508, 5,554,389, 5,993,844, 6,206,931, 6,099,567, and 8,192,763.

Submucosa-containing d-ECM tissue layers or other d-ECM tissue layers, when used in embodiments herein, are preferably highly purified, for example, as described in U.S. Pat. No. 6,206,931 or U.S. Pat. No. 8,192,763. Thus, preferred d-ECM tissue layers will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. As well, preferred d-ECM tissue layers for use herein will have a native immunoglobulin A (IgA) level of no greater than about 20 μg/g, more preferably no greater than about 10 μg/g. These and additional properties of submucosal d-ECM tissue layers or other d-ECM tissue layers taught in U.S. Pat. No. 6,206,931 and/or U.S. Pat. No. 8,192,763 may be characteristic of any d-ECM tissue layer or layers used in embodiments of the present disclosure.

Submucosa-containing or other d-ECM tissue layers used herein may retain one or more growth factors native to the source tissue for the tissue layers, including but not limited to basic fibroblast growth factor (FGF-2), transforming growth factor beta (TGF-beta), epidermal growth factor (EGF), cartilage derived growth factor (CDGF), and/or platelet derived growth factor (PDGF). As well, submucosal or other d-ECM tissue layers used herein may retain other native bioactive agents such as but not limited to proteins, glycoproteins, proteoglycans, and glycosaminoglycans. For example, d-ECM tissue layers may include native heparin, native heparin sulfate, native hyaluronic acid, native fibronectin, native cytokines, and the like. Thus, generally speaking, a submucosal or other d-ECM tissue layer may retain one or more native bioactive components that induce, directly or indirectly, a cellular response such as a change in cell morphology, proliferation, growth, protein or gene expression.

Submucosa-containing (i.e. “submucosal”) or other d-ECM tissue layers can be obtained from any suitable organ or other tissue source, usually sources containing connective tissues. The d-ECM tissue layers will typically be membranous tissue layers that include abundant collagen, most commonly being constituted at least about 80% by weight collagen on a dry weight basis. Such naturally-derived d-ECM tissue layers will for the most part include collagen fibers that are non-randomly oriented, for instance occurring as generally uniaxial or multi-axial but regularly oriented fibers. When processed to retain native bioactive factors, the d-ECM tissue layers can retain these factors interspersed as solids between, upon and/or within the collagen fibers. Particularly desirable naturally-derived d-ECM tissue layers for use in the invention will include significant amounts of such interspersed, non-collagenous solids that are readily ascertainable under light microscopic examination with appropriate staining. Such non-collagenous solids can constitute a significant percentage of the dry weight of the d-ECM tissue layer in certain embodiments, for example at least about 1%, at least about 3%, and at least about 5% by weight in various forms.

A submucosa-containing or other d-ECM tissue layer used in the present invention may also exhibit an angiogenic character and thus be effective to induce angiogenesis in a host engrafted with the tissue layer. In this regard, angiogenesis is the process through which the body makes new blood vessels to generate increased blood supply to tissues. Thus, angiogenic materials, when contacted with host tissues, promote or encourage the formation of new blood vessels into the materials. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

Further, in addition or as an alternative to the inclusion of native bioactive components, nonnative bioactive components such as those synthetically produced by recombinant technology or other methods (e.g., genetic material such as DNA), may be incorporated into a d-ECM tissue layer or layers used herein. These non-native bioactive components may be naturally-derived or recombinantly produced proteins that correspond to those natively occurring in an ECM tissue, but perhaps of a different species. These non-native bioactive components may also be drug substances. Illustrative drug substances that may be added to materials include, for example, anti-clotting agents, e.g. heparin, antibiotics, anti-inflammatory agents, thrombus-promoting substances such as blood clotting factors, e.g., thrombin, fibrinogen, and the like, and anti-proliferative agents, e.g. taxol derivatives such as paclitaxel. Such non-native bioactive components can be incorporated into and/or onto a d-ECM tissue layer in any suitable manner, for example, by surface treatment (e.g., spraying) and/or impregnation (e.g., soaking), just to name a few. Also, these substances may be applied to the d-ECM tissue layer in a premanufacturing step, immediately prior to the procedure (e.g., by soaking the tissue layer in a solution containing a suitable antibiotic such as cefazolin), or during or after engraftment of the tissue layer in a patient.

In certain forms, devices herein include a d-ECM tissue layer or layers receptive to tissue ingrowth. Upon implantation of such devices in accordance with the present disclosure, cells from the patient can infiltrate the d-ECM tissue layer(s), leading to new tissue growth on, around, and/or within the d-ECM tissue layer(s). In some embodiments, the d-ECM tissue layer or layers is/are remodelable and are capable of being absorbed and replaced by new tissue in the patient. Remodelable d-ECM tissue layers having a relatively more open matrix structure (i.e., higher porosity) are capable of exhibiting different material properties than those having a relatively more closed or collapsed matrix structure. For example, a d-ECM tissue layer having a relatively more open matrix structure is generally softer and more readily compliant to an implant site than one having a relatively more closed matrix structure. Also, the rate and amount of tissue growth in and/or around a remodelable d-ECM tissue layer can be influenced by a number of factors, including the amount of open space available in the material's matrix structure for the infusion and support of a patient's tissue-forming components, such as fibroblasts. Therefore, a more open matrix structure can provide for quicker, and potentially more, growth of patient tissue in and/or around a remodelable d-ECM tissue layer or layers, which in turn, can lead to quicker replacement of the d-ECM tissue layer or layers by patient tissue. In certain aspects, a d-ECM tissue layer or a multilayer laminate of a plurality of d-ECM tissue layers includes an open matrix structure formed by lyophilization drying of the layer or multilayer laminate.

In certain embodiments, as discussed above, the layer component 20 and/or the layer component 22 can be a laminate including two or more d-ECM tissue layers bonded together, for example two to four d-ECM tissue layers bonded together. As well, as discussed above, in the preparation of implant devices from layer component 20 and layer component 22, regions of respective d-ECM tissue layers of component 20 and component 22 are interfacially bonded to one another. In this regard, a variety of techniques for bonding surfaces of d-ECM tissue layers together can be used in preparing laminates for component 20 and/or 22 or in bonding regions of component 20 to component 22. These include, for instance, dehydrothermal bonding under heated, non-heated or lyophilization conditions, using adhesives, glues or other bonding agents, crosslinking with chemical agents, or any combination of these with each other or other suitable methods. The total thickness of a laminate of two or more bonded d-ECM layers when used as or in the layer component 20 and/or the layer component 22 can be in the range of about 200 microns to about 4,000 microns, or in the range of about 200 microns to about 1,000 microns, in certain forms. In preferred forms, the bonded d-ECM layers of a laminate used as or in layer component 20 and/or layer component 22, and/or the selected, interfacially bonded regions between the respective d-ECM layers of component 20 and component 22, will have surfaces of the d-ECM tissue layers directly contacting and dehydrothermally bonded to one another. As discussed herein, this dehydrothermal bonding can be resultant of and characterized by contacting the surfaces of the d-ECM tissue layers with one another while wet and dehydrating the surfaces while maintaining contact between them. For example, dehydration of the d-ECM tissue layers in contact with one another can effectively bond the surfaces to one another, even in the absence of other agents for achieving a bond. With sufficient dehydrothermal bonding to one another, the two or more d-ECM tissue layers can be caused to form a generally unitary collagenous structure. Some particularly useful dehydrating conditions for dehydrothermal bonding of surfaces of d-ECM tissue layers to one another include lyophilization conditions, evaporative cooling conditions, or vacuum-pressing conditions (e.g. where the d-ECM layers are compressed against one another during vacuum drying within a vacuum bag).

Additional aspects of the present disclosure provide medical products that include an implant device, for example a pocket implant device and/or a polymeric drug depot structure as described herein, in a sealed medical package. In some forms, such medical products include the medical implant and/or drug depot structure in sterile condition within the medical packaging. Illustratively, such a medical product can have packaging including a backing layer and a front film layer that are joined by a boundary of pressure-adhesive as is conventional in medical packaging, wherein the contents of the packaging are sealed between the backing layer and front film layer. Sterilization of such a medical product may be achieved, for example, by irradiation, ethylene oxide gas, or any other suitable sterilization technique, and the materials and other properties of the medical packaging will be selected accordingly.

For the purpose of promoting a further understanding of certain aspects of the present disclosure, the following specific Example is provided. It will be understood that this Example is illustrative, and not limiting, of embodiments of the present disclosure.

### Example

A first d-ECM tissue layer, in the form of a water-wetted, generally rectangular decellularized small intestinal submucosal tissue layer, was laid and spread flat upon a preparative surface. An elongate separating strip made of Tyvek® film, which is permeable to water vapor, was laid atop the first d-ECM tissue layer in a central location. The separating strip was longer than the first d-ECM tissue layer, and ends of the strip extended beyond opposite edges of the first d-ECM tissue layer. Subsequently, a second d-ECM tissue layer, in the form of a water-wetted, generally rectangular decellularized small intestinal submucosal tissue layer, was laid and spread flat overtop the separating strip and first d-ECM tissue layer. The second d-ECM layer was smoothed and pressed against the underlying materials to remove or minimize air bubbles that might be present between them. The resulting layup was placed in a freezer and allowed to freeze. The frozen layup was then placed in a lyophilizer and lyophilized to dryness, after which it was removed from the lyophilizer and the separating strip pulled out from between the first and second d-ECM layers. This provided a dried multilayer construct having a channel-form chamber region extending from edge to opposite edge in the construct, flanked on each side by dehydrothermally interfacially bonded regions of the first and second d-ECM layers.

The dried multilayer construct was then cut about its periphery to a desired shape for continued manufacture, and also to form a plurality of thru-holes. First and second predetermined portions of the channel-form chamber region for receiving respective first and second polymeric depot structures were left without any thru-holes. A first generally ring-shaped polymeric drug depot structure was slid into the channel-form chamber from one edge of the construct and moved centrally a distance in the chamber, and a second generally ring-shaped polymeric drug depot structure was slid into the channel from the opposite edge and moved centrally a distance in the chamber, such that the drug depot structures were each in their predetermined positions separated a distance from one another in a central region of the construct along which it was later to be folded. The drug depot structures were generally of the design depicted and described in conjunction with FIGS. 13-15, and were formed of polylactic acid/glycolic acid copolymer incorporating rifampin and minocycline. The drug depot structures were temporarily immobilized with locating pins. A sewing machine was then used to form a generally rectangular line of lock stitches positioned closely outside of and surrounding the periphery of each of the drug depot structures, with a first side and a second side of the stitched rectangle each traversing and closing the channel-form chamber on opposite sides of the surrounded drug depot structure. This confined each of the drug depot structures to a smaller chamber space within the original larger channel-form chamber. The stitching material used was an absorbable polyglycolic acid copolymer suture (TRISORB® suture). The stitched construct was then folded in half generally as shown in FIGS. 6 and 7, to overlap one half of the construct with another half of the construct, with the first and second polymeric drug depot structures aligned and overlapping with one another. A sewing machine was used to form a continuous line of lock stitches connecting the overlapped first and second halves of the construct to one another near the periphery of three sides of the construct. The shape of the line of lock stitches connecting the two halves was generally as shown in FIG. 8. The construct was then cut to a desired final shape for the implantable medical pocket device.

While embodiments of the disclosure have been illustrated and described in detail in the drawings and foregoing description, the same is to be considered as illustrative and not restrictive in character, it being understood that only some embodiments have been shown and described and that all changes and modifications that come within the spirit of the disclosures herein are desired to be protected. As examples, the following Listing of Certain Disclosed Embodiments, provided as clauses, provides an identification of some of the embodiments disclosed herein. It will be understood that this listing is non-limiting, and that individual features or combinations of features (e.g. 2, 3 or 4 features) as described in the Detailed Description above can be combined with the below-listed, enumerated Embodiments of the Listing of Certain Disclosed Embodiments to provide additional disclosed embodiments herein.

### LISTING OF CERTAIN DISCLOSED EMBODIMENTS

1. A method for making a drug depot-bearing medical implant, comprising:
providing a first multilayer structure in which a first decellularized extracellular matrix (d-ECM) tissue layer is interfacially bonded to a second d-ECM tissue layer in selected regions to provide a first sidewall chamber between unbonded regions of the first and second d-ECM tissue layers, wherein the first multilayer structure defines an opening to the first sidewall chamber;
providing a first polymeric drug depot structure in the first sidewall chamber; and
securing the first polymeric drug depot structure in the first sidewall chamber.

2. The method of clause 1, wherein said securing comprises closing the opening.

3. The method of clause 2, wherein said closing includes applying a stitch line across the opening.

4. The method of any one of clauses 1 to 3, wherein said securing includes applying a stitch line that entirely circumferentially surrounds the first polymeric drug depot structure.

5. The method of any one of clauses 1 to 4, wherein said securing comprises adhering the first polymeric drug depot structure to the first d-ECM tissue layer and/or the second d-ECM tissue layer.

6. The method of any one of clauses 1 to 5, wherein said providing a first multilayer structure includes:
providing a multilayer layup including the first d-ECM tissue layer a wetted state and the second d-ECM tissue layer in a wetted state in an overlapped condition, the layup having a first layup region in which a first surface portion of the first d-ECM tissue layer is separated from the first face portion of the second d-ECM tissue layer by a spacer member in a position therebetween, a second layup region in which a second surface portion of the first d-ECM tissue layer is received against a second surface portion of the second d-ECM tissue layer, and a third layup region in which a third surface portion of the first d-ECM tissue layer is received against a third surface portion of the second d-ECM tissue layer, wherein the first layup region is between the second layup region and the third layup region;
drying the multilayer layup so as to dehydrothermally bond the second surface portions to one another and the third surface portions to one another while leaving the first surface portions unbonded to one another; and
removing the spacer member from the position between the first face portions of the first and second layers.

7. The method of any one of clauses 1 to 6, also comprising:
forming an implantable medical pocket device including a first pocket sidewall, a second pocket sidewall, a pocket chamber between the first pocket sidewall and the second pocket sidewall, and a pocket opening to the pocket chamber, wherein the first pocket sidewall includes at least a first portion of the multilayer structure that includes the first sidewall chamber and the first polymeric drug depot structure.

8. The method of clause 7, wherein the multilayer structure includes the first portion and a second portion, the method also comprising:
folding the multilayer structure to provide a folded structure having the first portion overlapped with the second portion, the first portion positioned to provide the first pocket sidewall and the second portion positioned to provide the second pocket sidewall; and
connecting the first portion to the second portion along a connection path to create the pocket chamber.

9. The method of any one of clauses 1 to 8, wherein:
the multilayer structure also includes a second chamber between unbonded regions of the first and second d-ECM tissue layers and defines an opening to the second chamber; and
the method also includes inserting a second polymeric drug depot structure through the opening and into to the second chamber, and securing the second polymeric drug depot structure in the second chamber.

10. The method of clause 8, wherein the second portion of the multilayer structure includes a second chamber between unbonded regions of the first and second d-ECM tissue layers and defines an opening to the second chamber, the method also including:
inserting a second polymeric drug depot structure through the opening to the second chamber and into the second chamber; and
securing the second polymeric drug depot structure in the second chamber.

11. The method of any one of clauses 8 to 10, also comprising cutting the folded structure to provide an outer peripheral shape of the implantable medical pocket device.

12. The method of clause 11, wherein said outer peripheral shape includes a peripheral edge portion outward of and extending generally parallel to at least a portion of the connection path.

13. The method of any preceding clause, wherein the first polymeric drug depot structure comprises a ribbon of polymeric material extending in a closed path to define an inner opening, with an inner edge of the ribbon bounding the inner opening and an outer edge of the ribbon defining an outer periphery of the first polymeric drug depot structure.

14. The method of any preceding clause, wherein the multilayer structure includes a third d-ECM tissue layer interfacially bonded to the first d-ECM tissue layer and a fourth d-ECM tissue layer interfacially bonded to the second d-ECM tissue layer, wherein the first and second d-ECM tissue layers are between the third and fourth d-ECM tissue layers in the multilayer structure.

15. The method of any preceding clause, wherein the first polymeric drug depot structure contains at least one antibiotic agent.

16. The method of clause 15, wherein the first polymeric drug depot structure contains rifampin.

17. The method of clause 15 or 16, wherein the first polymeric drug depot structure contains minocycline.

18. An implantable medical pocket device, comprising:
a first pocket sidewall, a second pocket sidewall, and a pocket chamber between the first pocket sidewall and the second pocket sidewall; and
a first polymeric drug depot structure;
   wherein:
(i) the first polymeric drug depot structure is positioned in a first sidewall chamber of the first pocket sidewall between a first decellularized extracellular matrix (d-ECM) tissue layer and second d-ECM tissue layer interfacially bonded to one another in selected regions to provide the first sidewall chamber between unbonded regions of the first and second d-ECM tissue layers; or
(ii) the first polymeric drug depot attaches the first pocket sidewall to the second pocket sidewall.

19. The device of clause 18, wherein the first polymeric drug depot structure is positioned in a first sidewall chamber of the first pocket sidewall between a first decellularized extracellular matrix (d-ECM) tissue layer and second d-ECM tissue layer interfacially bonded to one another in selected regions to provide the first sidewall chamber between unbonded regions of the first and second d-ECM tissue layers.

20. The device of claim 19, also comprising a stitch line closing an opening to the first sidewall chamber to secure the first polymeric drug depot structure in the first sidewall chamber, optionally wherein the stitch line entirely circumferentially surrounds the first polymeric drug depot structure.

21. The device of clause 19 or 20, wherein said first polymeric drug depot structure is adhered to the first d-ECM tissue layer and/or the second d-ECM tissue layer.

22. The device of any one of clauses 19 to 21, wherein the first d-ECM tissue layer is interfacially bonded directly against and to the second d-ECM tissue layer in the selected regions by dehydrothermal bonding.

23. The device of any one of clauses 19 to 22, wherein:
the second pocket sidewall also includes first and second decellularized extracellular matrix (d-ECM) tissue layers interfacially bonded to one another in selected regions to provide a second sidewall chamber between unbonded regions of the first and second d-ECM tissue layers of the second pocket sidewall; and
a second polymeric drug depot structure is positioned in the second sidewall chamber.

24. The device of any one of clauses 19 to 23, comprising a stitch line extending in a stitch path and attaching the first pocket sidewall to the second pocket sidewall to create the pocket chamber.

25. The device of clause 24, including an outer peripheral edge positioned outward of and extending generally parallel to at least a portion of the stitch path.

26. The device of any one of clauses 19 to 25, wherein the first polymeric drug depot structure includes a ribbon of polymeric material extending in a closed path to define an inner opening, with an inner edge of the ribbon bounding the inner opening and an outer edge of the ribbon defining an outer periphery of the first polymeric drug depot structure.

27. The device of any one of clauses 19 to 26, wherein:

the first pocket sidewall includes a third d-ECM tissue layer interfacially bonded to the first d-ECM tissue layer and a fourth d-ECM tissue layer interfacially bonded to the second d-ECM tissue layer, with the first and second d-ECM layers positioned between the third and fourth d-ECM layers.

28. The device of any one of clauses 18 to 27, wherein the first polymeric drug depot structure contains at least one antibiotic agent.

29. The device of clause 28, wherein the at least one antibiotic agent includes rifampin.

30. The device of clause 28 or 29, wherein the at least one antibiotic agent includes minocycline.

31. The device of any one of clauses 19 to 30, wherein the d-ECM tissue is a submucosal d-ECM tissue.

32. The device of clause 31, wherein the submucosal d-ECM tissue is small intestinal submucosal d-ECM tissue.

33. The device of clause 31, wherein the submucosal d-ECM tissue is small intestinal submucosal d-ECM tissue, urinary bladder submucosal d-ECM tissue, or stomach submucosal d-ECM tissue.

34. The device of any one of clauses 31 to 33, wherein the submucosal d-ECM tissue is a porcine, ovine, bovine, or caprine d-ECM tissue.

35. The device of claim 18, wherein the first polymeric drug depot attaches the first pocket sidewall to the second pocket sidewall, optionally wherein the first pocket sidewall and/or the second pocket sidewall incorporates at least one d-ECM tissue layer.

36. A method for preparing an implant combination for insertion in a patient, comprising:
inserting a medical implant into the pocket chamber of an implantable medical pocket device according to any one of clauses 18 to 35.

37. The method of clause 36, wherein the medical implant is a pulse generator of an electronic medical implant.

38. The method of clause 37, wherein the electronic medical implant is a cardiac electronic medical implant or a neurostimulator medical implant.

39. The method of clause 38, wherein the cardiac electronic medical implant is a cardiac defibrillator and/or a cardiac pacemaker.

40. The method of any one of clauses 36 to 39, which is also for treating the patient, the method also comprising inserting into an implant site in the patient the implantable medical pocket device with the medical implant received in the pocket chamber.

41. The method of clause 40, wherein said implant site is a subcutaneous implant site.

42. A polymeric drug depot structure, comprising:
a ribbon of polymeric material incorporating one or more drugs, the ribbon extending in a closed path to define an inner opening, with an inner edge of the ribbon bounding the inner opening and an outer edge of the ribbon defining an outer periphery of the polymeric drug depot structure.

43. The drug depot structure of clause 42, wherein the inner edge and the outer edge extend along paths concentric to one another.

44. The drug depot structure of clause 42 or 43, wherein the ribbon has a maximum thickness in the range of about 0.1 mm to about 3 mm, or about 0.2 mm to about 1 mm.

45. The drug depot structure of any one of clauses 42 to 44, wherein the ribbon has a minimum thickness of about 0.1 mm, or about 0.2 mm.

46. The drug depot structure of any one of clauses 42 to 45, wherein the ribbon has a substantially constant thickness.

47. The drug depot structure of any one of clauses 42 to 46, wherein the ribbon has a minimum width in the range of about 1 mm to about 15 mm, or about 2 mm to about 10 mm, or about 3 mm to about 7 mm, wherein the width is taken as the smallest distance between the inner edge and the outer edge.

48. The drug depot structure of any one of clauses 42 to 47, wherein the ribbon has a maximum width of about 15 mm, or about 10 mm.

49. The drug depot structure of any one of clauses 42 to 48, wherein the ribbon has a substantially constant width, wherein the width is taken as the smallest distance between the inner edge and the outer edge.

50. The drug depot structure of any one of clauses 42 to 49, wherein the ribbon has a ratio of a maximum width of the ribbon to a maximum thickness of the ribbon in the range of about 150:1 to about 5:1, or about 50:1 to about 20:1, wherein the width is taken as the smallest distance between the inner edge and the outer edge.

51. The drug depot structure of any one of clauses 42 to 50, wherein the inner edge and the outer edge extend along concentric circular, concentric ovoid, or concentric polygonal paths, with respect to one another.

52. The drug depot structure of any one of clauses 42 to 51, wherein the outer edge of the ribbon adjoins a first side surface of the ribbon in a radiused corner.

53. The drug depot structure of any one of clauses 42 to 51, wherein the inner edge of the ribbon adjoins a first side surface of the ribbon in a radiused corner.

54. The drug depot structure of clause 52, wherein the inner edge of the ribbon adjoins the first side surface of the ribbon in a radiused corner.

55. The drug depot structure of any one of clauses 52 to 54, wherein the outer edge of the ribbon adjoins a second side surface of the ribbon in a non-radiused corner, the second side surface being opposite the first side surface.

56. The drug depot structure of any one of clauses 52 to 54, wherein the inner edge of the ribbon adjoins a second side surface of the ribbon in a non-radiused corner, the second side surface being opposite the first side surface.

57. The drug depot structure of clause 55, wherein the inner edge of the ribbon adjoins the second side surface of the ribbon in a non-radiused corner.

58. A polymeric drug depot structure, comprising:
a polymeric material layer incorporating one or more drugs, wherein the polymeric material layer has a minimum thickness of about 0.1 mm and a maximum thickness of about 3 mm, or wherein the polymeric material layer has a minimum thickness of about 0.1 mm and a maximum thickness of about 1 mm.

59. The drug depot structure of any one of clauses 42 to 58, wherein the depot structure is flexible.

60. The drug depot structure of any one of clauses 42 to 59, wherein the depot structure exhibits flexibility to conform to patient tissue when pressed thereagainst.

61. The drug depot structure of any one of clauses 42 to 60, wherein the one or more drugs includes one or more antibiotic agents.

62. The drug depot structure of clause 61, wherein the one or more antibiotic agents includes rifampin.

63. The drug depot structure of clause 61 or 62, wherein the one or more antibiotic agents includes minocycline.

64. The drug depot structure of any one of clauses 42 to 63, wherein the polymeric material is absorbable.

65. The drug depot structure of clause 64, wherein the polymeric material includes at least one member selected from polycaprolactone polymers, polyglactin polymers, polydioxanone polymers, polylactic acid polymers, polylactide polymers, polylactic acid/glycolic acid copolymers.

66. The drug depot structure of clause 65, wherein the polymeric material includes a polylactic acid/glycolic acid copolymer.

67. The drug depot structure of clause 66, wherein the polymeric material consists of a polylactic acid/glycolic acid copolymer.

68. A medical implant comprising at least one polymeric drug depot structure according to any one of clauses 42 to 67.

69. The medical implant of clause 68, which is an implantable medical pocket device.

70. The medical implant of clause 69, wherein the pocket device includes a first pocket sidewall, a second pocket sidewall, and a pocket chamber between the first and second pocket sidewalls, and wherein the first pocket sidewall incorporates the at least one polymeric drug depot structure.

71. The medical implant of clause 70, wherein the at least one polymer drug depot structure includes at least first and second polymeric drug depot structures according to any one of clauses 42 to 67, wherein the first pocket sidewall incorporates the first polymeric drug depot structure and the second pocket sidewall incorporates the second polymeric drug depot structure.

72. The medical implant of clause 70, wherein the at least one polymeric drug depot structure is positioned in a chamber occurring between first and second layer components of the first sidewall.

73. The medical implant of clause 71, wherein the first polymeric drug depot is positioned in a first chamber occurring between first and second layer components of the first sidewall and the second polymeric drug depot structure is positioned in a second chamber occurring between first and second layer components of the second sidewall.

74. The medical implant of any one of clauses 70 to 73, wherein the first pocket sidewall includes at least a first decellularized extracellular matrix (d-ECM) tissue layer and the second pocket sidewall includes at least a second d-ECM tissue layer.

75. A method according to any one of clauses 1 to 17 or a device according to any one of clauses 18 to 35, wherein the first polymeric depot structure and, when present, the second polymeric drug depot structure, is a polymeric drug depot structure according to any one of clauses 42 to 67.

76. A method for making a drug depot-bearing medical implant, comprising:
attaching a first pocket sidewall to a second pocket sidewall with a first polymeric drug depot structure to form a pocket chamber between the first pocket sidewall and the second pocket sidewall.

The uses of the terms “a” and “an” and “the” and similar references herein (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the products or methods defined by the claims.

All references cited herein are indicative of the level of skill in the art and are hereby incorporated by reference in their entirety.

1. A method for making a drug depot-bearing medical implant, comprising:
providing a first multilayer structure in which a first decellularized extracellular matrix (d-ECM) tissue layer is interfacially bonded to a second d-ECM tissue layer in selected regions to provide a first sidewall chamber between unbonded regions of the first and second d-ECM tissue layers, wherein the first multilayer structure defines an opening to the first sidewall chamber;
providing a first polymeric drug depot structure in the first sidewall chamber; and
securing the first polymeric drug depot structure in the first sidewall chamber.

2. The method of claim 1, wherein said securing comprises closing the opening.

3. The method of claim 2, wherein said closing includes applying a stitch line across the opening.

4. The method of claim 1, wherein said securing includes applying a stitch line that entirely circumferentially surrounds the first polymeric drug depot structure.

5. The method of claim 1, wherein said securing comprises adhering the first polymeric drug depot structure to the first d-ECM tissue layer and/or the second d-ECM tissue layer.

6. The method of claim 1, wherein said providing a first multilayer structure includes:
providing a multilayer layup including the first d-ECM tissue layer a wetted state and the second d-ECM tissue layer in a wetted state in an overlapped condition, the layup having a first layup region in which a first surface portion of the first d-ECM tissue layer is separated from the first face portion of the second d-ECM tissue layer by a spacer member in a position therebetween, a second layup region in which a second surface portion of the first d-ECM tissue layer is received against a second surface portion of the second d-ECM tissue layer, and a third layup region in which a third surface portion of the first d-ECM tissue layer is received against a third surface portion of the second d-ECM tissue layer, wherein the first layup region is between the second layup region and the third layup region;
drying the multilayer layup so as to dehydrothermally bond the second surface portions to one another and the third surface portions to one another while leaving the first surface portions unbonded to one another; and
removing the spacer member from the position between the first face portions of the first and second layers.

7. The method of claim 1, also comprising:
forming an implantable medical pocket device including a first pocket sidewall, a second pocket sidewall, a pocket chamber between the first pocket sidewall and the second pocket sidewall, and a pocket opening to the pocket chamber, wherein the first pocket sidewall includes at least a first portion of the multilayer structure that includes the first sidewall chamber and the first polymeric drug depot structure.

8. The method of claim 7, wherein the multilayer structure includes the first portion and a second portion, the method also comprising:
folding the multilayer structure to provide a folded structure having the first portion overlapped with the second portion, the first portion positioned to provide the first pocket sidewall and the second portion positioned to provide the second pocket sidewall; and
connecting the first portion to the second portion along a connection path to create the pocket chamber.

9. The method of claim 1, wherein:
the multilayer structure also includes a second chamber between unbonded regions of the first and second d-ECM tissue layers and defines an opening to the second chamber; and
the method also includes inserting a second polymeric drug depot structure through the opening and into to the second chamber, and securing the second polymeric drug depot structure in the second chamber.

10-12. (canceled)

13. The method of claim 1, wherein the first polymeric drug depot structure comprises a ribbon of polymeric material extending in a closed path to define an inner opening, with an inner edge of the ribbon bounding the inner opening and an outer edge of the ribbon defining an outer periphery of the first polymeric drug depot structure.

14. The method of claim 1, wherein the multilayer structure includes a third d-ECM tissue layer interfacially bonded to the first d-ECM tissue layer and a fourth d-ECM tissue layer interfacially bonded to the second d-ECM tissue layer, wherein the first and second d-ECM tissue layers are between the third and fourth d-ECM tissue layers in the multilayer structure.

15. The method of claim 1, wherein the first polymeric drug depot structure contains at least one antibiotic agent.

16-17. (canceled)

18. An implantable medical pocket device, comprising:
a first pocket sidewall, a second pocket sidewall, and a pocket chamber between the first pocket sidewall and the second pocket sidewall; and
a first polymeric drug depot structure;
wherein:
(i) the first polymeric drug depot structure is positioned in a first sidewall chamber of the first pocket sidewall between a first decellularized extracellular matrix (d-ECM) tissue layer and second d-ECM tissue layer interfacially bonded to one another in selected regions to provide the first sidewall chamber between unbonded regions of the first and second d-ECM tissue layers; or
(ii) the first polymeric drug depot structure attaches the first pocket sidewall to the second pocket sidewall.

19. The device of claim 18, wherein the first polymeric drug depot structure is positioned in a first sidewall chamber of the first pocket sidewall between a first decellularized extracellular matrix (d-ECM) tissue layer and second d-ECM tissue layer interfacially bonded to one another in selected regions to provide the first sidewall chamber between unbonded regions of the first and second d-ECM tissue layers.

20. The device of claim 19, also comprising a stitch line closing an opening to the first sidewall chamber to secure the first polymeric drug depot structure in the first sidewall chamber, optionally wherein the stitch line entirely circumferentially surrounds the first polymeric drug depot structure.

21. The device of claim 19, wherein said first polymeric drug depot structure is adhered to the first d-ECM tissue layer and/or the second d-ECM tissue layer.

22. The device of claim 19, wherein the first d-ECM tissue layer is interfacially bonded directly against and to the second d-ECM tissue layer in the selected regions by dehydrothermal bonding.

23. The device of claim 19, wherein:
the second pocket sidewall includes first and second decellularized extracellular matrix (d-ECM) tissue layers interfacially bonded to one another in selected regions to provide a second sidewall chamber between unbonded regions of the first and second d-ECM tissue layers; and
a second polymeric drug depot structure is positioned in the second sidewall chamber.

24. The device of claim 19, comprising a stitch line extending in a stitch path and attaching the first pocket sidewall to the second pocket sidewall to create the pocket chamber.

25. The device of claim 24, including an outer peripheral edge positioned outward of and extending generally parallel to at least a portion of the stitch path.

26. The device of claim 19, wherein the first polymeric drug depot structure includes a ribbon of polymeric material extending in a closed path to define an inner opening, with an inner edge of the ribbon bounding the inner opening and an outer edge of the ribbon defining an outer periphery of the first polymeric drug depot structure.

27. The device of claim 19, wherein:
the first pocket sidewall includes a third d-ECM tissue layer interfacially bonded to the first d-ECM tissue layer and a fourth d-ECM tissue layer interfacially bonded to the second d-ECM tissue layer, with the first and second d-ECM layers of the first pocket sidewall positioned between the third and fourth d-ECM layers of the first pocket sidewall.

28. The device of claim 18, wherein the first polymeric drug depot structure contains at least one antibiotic agent.

29-30. (canceled)

31. The device of claim 19, wherein the d-ECM tissue is a submucosal d-ECM tissue.

32-35. (canceled)

36. A method for preparing an implant combination for insertion in a patient, comprising:
inserting a medical implant into the pocket chamber of an implantable medical pocket device according to claim 18.

37-41. (canceled)
